(19) United States
(12) Patent Application Publication
(10) Pub. No.: US 2025/0198997 A1
(43) Pub. Date: Jun. 19, 2025

(54) MAGNETIC PARTICLE-BASED IMMUNOASSAY AND METHODS OF USING THE SAME

(71) Applicant: Vital Biosciences Inc., Kitchener (CA)

(72) Inventors: Andrew WARD, Everett, MA (US); ArunRichard CHANDRASEKARAN, Somerville, MA (US); Daniel CHEN, Cambridge, MA (US); Christopher BLANCHARD, Somerville, MA (US); Padric GARDEN, Cambridge, MA (US); Bradley DEMARCO, Somerville, MA (US); Joshua FORMAN, Winchester, MA (US); Mounir A. KOUSSA, Kitchener (CA); Lisa CALDWELL, Westford, MA (US)

(21) Appl. No.: 19/053,713

(22) Filed: Feb. 14, 2025

Related U.S. Application Data
(63) Continuation of application No. 16/750,629, filed on Jan. 23, 2020, now abandoned.
Application No. 16/750,629 is a continuation of application No. 16/479,827, filed on Jul. 22, 2019.
(60) Provisional application No. 62/554,393, filed on Sep. 5, 2017.
(60) Provisional application No. 62/450,623, filed on Jan. 26, 2017.

(51) Int. Cl. G01N 33/543; G01N 33/58; G01N 35/00

(52) U.S. Cl. CPC G01N 33/54326; G01N 33/588; G01N 35/0098

(57) ABSTRACT

The invention describes, in part, improved methods, assays, and kits for detecting analytes in biological samples with magnetic particles.

### CROSS-REFERENCE TO RELATED APPLICATIONS

The international application claims the benefit of U.S. Provisional Application No. 62/450,623, filed Jan. 26, 2017, and U.S. Provisional Application No. 62/544,393, filed Aug. 11, 2017, the entirety of which are incorporated herein by reference.

### FIELD OF THE INVENTION

The invention described herein relates generally to improved methods, assays, and kits for detecting analytes in biological samples.

### BACKGROUND OF THE INVENTION

Analyte detection has various clinical and non-clinical applications in industries ranging from medicine and biological research to environmental science and beyond. Traditional methods for analyte detection involve assays such as enzyme-linked immunosorbent assays (ELISA), mass spectrometry, and high pressure liquid chromatography (HPLC). While HPLC and mass spectrometry may be used to detect analytes on the basis of charge and/or size, ELISA may be used to detect an analyte based on antigens on the analyte that are recognizable by capture and detection agents (e.g., antibodies, aptamers, etc.). In particular, ELISA assay has become a relatively common detection method utilized in the life sciences. However, conventional ELISA may be time-consuming as it involves various incubation and washing steps and may not provide sufficient sensitivity for various applications. Further, the parameters for carrying out ELISA assays are highly variable thus rendering the assay difficult to develop as a universal platform, particularly as home diagnostics for individual users.

Indeed, healthcare would benefit greatly from the ability to monitor biomarkers easily and frequently at home. For instance, individuals are better suited to monitor their health status and direct their care than medical professionals, who faced limited time and resources for treating patients. Further, empowered with health information, there is the capacity for more accurate life planning, including, for instance, timing of reproduction.

Accordingly, there remains a need for improved methods for detecting an analyte in a sample that takes less time and input to perform compared to conventional methods, while maintaining or improving the sensitivity of detection.

### SUMMARY OF THE INVENTION

Immunoassays, kits including such assays, and methods of using the same to detect the presence, absence, or level of an analyte of interest in a sample are described herein.

In some aspects, the invention relates to systems and methods for detecting analytes, e.g. antigens from biological samples, with improved sensitivity than presently available methods, for instance relying on antibody-based detection of an antigen of interest, e.g. one that is useful for correlating with an individual's health state, with magnetic-mediated separation. The immunoassays, kits, and methods described herein provide considerable advantages over immunoassays in the field. Indeed, in some embodiments, the invention described herein includes a lack of sample preparation that is not found in known immunoassays. Current methods in the field require significant sample preparation. Here, in some embodiments, samples may be combined with the necessary reagents, which include a magnetic conjugate and a reporter, or reporter conjugate, in an analysis chamber and a magnetic field may be applied as a “pull down” step, followed by visualization and/or quantification of the reporter.

In an embodiment, the invention includes a method for detecting the presence, absence, or level of an analyte of interest in a sample. In some embodiments, the sample may be a bodily fluid, as defined herein.

In an embodiment, the methods of the invention may include the step of adding sample to an analysis chamber. In some embodiments, adding sample to the analysis chamber may include delivering the sample to a sample collector (e.g., an absorbent or wicking material) in fluid communication with the analysis chamber. In some embodiments, the sample collector may then feed the sample into the analysis chamber. In some embodiments, the methods of the invention may include contacting the sample with a magnetic conjugate comprising a magnetic particle and a capture moiety configured to bind the analyte of interest in the sample. In some embodiments, the magnetic conjugate may be disposed at the sample collector and the step of contacting the sample with the magnetic conjugate may occur at the sample collector. In some embodiments, the magnetic conjugate may be imbedded in a portion of the sample collector before a sample is added to the sample collector. In some embodiments, the methods of the invention may include contacting the sample in the analysis chamber with a magnetic conjugate. In some embodiments, the capture moiety is an antibody, an antigen-binding fragment, an antigen, a receptor, a ligand, an aptamer, an aptamer receptor, a nucleic acid, or a small molecule. In some embodiments, the capture moiety is a capture antibody. In some embodiments, the magnetic conjugate comprises a magnetic particle and a capture antibody.

In some embodiments, the methods described herein may include the step of contacting the sample with a reporter or a reporter conjugate comprising a reporter and a reporter binding moiety configured to bind the analyte of interest in the sample. In some embodiments, the reporter or reporter conjugate may be disposed at the sample collector and the step of contacting the sample with the reporter or reporter conjugate may occur at the sample collector. In some embodiments, the reporter or reporter conjugate may be imbedded in a portion of the sample collector before a sample is added to the sample collector. In some embodiments, the methods described herein may include the step of contacting the sample in the analysis chamber with a reporter or a reporter conjugate. In some embodiments, the reporter binding moiety is an antibody, an antigen-binding fragment, an antigen, a receptor, a ligand, an aptamer, an aptamer receptor, a nucleic acid, or a small molecule. In some embodiments, the reporter binding moiety is a reporter antibody. In some embodiments, the reporter conjugate comprises a reporter and a reporter antibody. In some embodiments, the methods described herein may include the step of binding the analyte of interest with the capture antibody and the reporter antibody. In some embodiments, the methods described herein may include the step of separating the analyte of interest from the sample by applying a magnetic field to the analysis chamber. In some embodiments, the methods described herein may include the step of detecting the presence, absence, or level of the analyte of interest by detecting the reporter.

In some embodiments, the reporter may include a metal core (e.g., a metal microparticle or metal nanoparticle) and may include a silica shell. In some embodiments, the reporter includes a metal core and has a silica shell. In some embodiments, the reporter includes a plurality of quantum dots. In some embodiments, the reporter includes a metal core with a silica shell and the silica shell is impregnated with a plurality of quantum dots. In some embodiments, the reporter includes a metal core and the metal core may comprise, or another metal as described herein.

In some embodiments, the reporter described herein may be a fluorescent reporter, a phosphorescent reporter, or a colorimetric reporter such as a colored particle that may be configured to measure absorbance or scattering of light (or, for example, the presence/absence of a certain color by colorimetric analysis).

In some embodiments, the methods described herein may further include the step of concentrating the analyte of interest in the sample by applying a magnetic field to the analysis chamber after contacting the sample with the magnetic conjugate; and then reducing the volume of the sample in the analysis chamber. In some embodiments, the method described herein may further include the step of deactivating the magnetic field before contacting the sample with the reporter conjugate.

In some embodiments, the methods described herein may further include the step of concentrating the analyte of interest in the sample by applying a magnetic field to the analysis chamber after contacting the sample with the magnetic conjugate; removing a volume of the sample from the analysis chamber; and adding a volume of buffer and/or an additional volume of the sample to the analysis chamber. In some embodiments, the methods described herein further include the step of deactivating the magnetic field before contacting the sample with the reporter conjugate.

In some embodiments, the reporter antibody described herein is labeled with biotin. In some embodiments, the reporter described herein is functionalized with streptavidin. In some embodiments, the reporter antibody described herein is labeled with streptavidin. In some embodiments, the reporter described herein is functionalized with biotin.

In some embodiments, the analyte of interest described herein may be any of the analytes and/or biomarkers described herein. In some embodiments, the analyte of interest may be selected from human chorionic gonadotropin (hCG), luteinizing hormone (LH)/Lutropin, prostate specific antigen (PSA), herpes simplex virus (HSV) antibodies, estrone-3-glucuronide (E3G), bacteria, hemoglobin AlC, C-reactive protein, an inflammation biomarker, troponin, lyme disease antigen, lyme disease antibodies, an LDL biomarker, an HDL biomarker, a total cholesterol biomarker, thyroid stimulating hormone, a hepatitis C virus biomarker, a rhino virus biomarker, an influenza virus biomarker, a liver function biomarker, estrogen, progesterone, lactic acid, and combinations thereof. In some embodiments, the bacteria may be Streptococcus-A, Chlamydia, and/or Gonorrhea. In some embodiments, the inflammation biomarker may be CRP, SAA, and/or MP8. In some embodiments, the liver function biomarker may be ALT and/or AST. In some embodiments, the analyte of interest may be selected from the group consisting of an ovulation biomarker, a pregnancy biomarker, a strep throat biomarker, a prostate cancer biomarker, a herpes biomarker, a diabetes biomarker, an inflammation biomarker, a heart attack biomarker, a chlamydia biomarker, a bacteria biomarker, a lyme disease biomarker, a cholesterol biomarker, a hypothydroidism biomarker, a hepatitis C biomarker, a rhino virus biomarker, an influenza biomarker, a liver function biomarker, a fertility biomarker, a muscle fatigue biomarker, and combinations thereof.

In some embodiments, an ovulation biomarker may be derived from a urine, blood, or serum based sample. In some embodiments, a pregnancy biomarker may be derived from a urine or blood based sample. In some embodiments, a strep throat biomarker may be derived from a saliva based sample. In some embodiments, a saliva based sample may be an aliquot of saliva, a cheek swab, or a throat swab. In some embodiments, a prostate cancer biomarker may be derived from a blood, serum, or urine based sample. In some embodiments, a herpes biomarker may be derived from a blood or saliva derived sample.

In an embodiment, the methods described herein may detect the presence, absence, or level of an analyte of interest in a sample.

In some embodiments, the methods described herein may include the steps of contacting a sample with a magnetic conjugate comprising a magnetic particle and a capture moiety configured to bind the analyte of interest in the sample; contacting the sample with a reporter conjugate comprising a reporter and a reporter binding moiety configured to bind the analyte of interest in the sample; binding the analyte of interest with the capture moiety and the reporter binding moiety; separating the analyte of interest from the sample by applying a magnetic field to the analysis chamber; and/or detecting the presence, absence, or level of the analyte of interest by detecting the reporter.

In some embodiments, the methods described herein may include the steps of contacting the sample with a reporter conjugate comprising a reporter and a reporter binding moiety configured to bind the analyte of interest in the sample; contacting a sample with a magnetic conjugate comprising a magnetic particle and a capture moiety configured to bind the analyte of interest in the sample; binding the analyte of interest with the capture moiety and the reporter binding moiety; separating the analyte of interest from the sample by applying a magnetic field to the analysis chamber; and/or detecting the presence, absence, or level of the analyte of interest by detecting the reporter.

In some embodiments, the methods described herein may include the steps of contacting a sample with a magnetic conjugate comprising a magnetic particle and a capture moiety configured to bind the analyte of interest in the sample; binding the analyte of interest with the capture moiety; separating the analyte of interest from the sample by applying a magnetic field to the analysis chamber to pull down the magnetic conjugates with analyte of interest associated therewith; contacting the sample with a reporter conjugate comprising a reporter and a reporter binding moiety configured to bind the analyte of interest in the sample; binding the analyte of interest with the reporter binding moiety; separating the analyte of interest with reporter binding moiety bound thereto from the sample by applying a magnetic field to the analysis chamber; and/or detecting the presence, absence, or level of the analyte of interest by detecting the reporter with a light source and photodetector.

In some embodiments, the methods described herein may include the steps of contacting a sample with a reporter conjugate comprising a reporter and a reporter binding moiety configured to bind the analyte of interest in the sample; binding the analyte of interest with the reporter binding moiety; contacting the sample with a magnetic particle-labeled analyte configured to bind the reporter conjugate in the absence of the analyte of interest in the sample; separating the magnetic particle-labeled analyte from the sample by applying a magnetic field to the sample; and/or detecting the presence, absence, or level of the analyte of interest by detecting the reporter.

In some embodiments, the methods described herein may include the steps of contacting a sample with a magnetic conjugate comprising a magnetic particle and a capture moiety configured to bind the analyte of interest in the sample; binding the analyte of interest with the capture moiety; contacting the sample with a reporter binding moiety comprising a biotin label configured to bind the analyte of interest in the sample; contacting the sample with a reporter comprising a streptavidin label configured to bind the biotin label; separating the analyte of interest from the sample by applying a magnetic field to the sample; and/or detecting the presence, absence, or level of the analyte of interest by detecting the reporter.

In some embodiments, the methods described herein may include the steps of contacting a sample with a magnetic conjugate comprising a magnetic particle and a capture moiety configured to bind the analyte of interest in the sample; binding the analyte of interest with the capture moiety; contacting the sample with a reporter binding moiety comprising a streptavidin label configured to bind the analyte of interest in the sample; contacting the sample with a reporter comprising a biotin label configured to bind the streptavidin label; separating the analyte of interest from the sample by applying a magnetic field to the sample; and/or detecting the presence, absence, or level of the analyte of interest by detecting the reporter.

In an embodiment, the methods described herein may include the steps of adding sample to an analysis chamber; contacting the sample with a magnetic conjugate comprising a magnetic particle and a capture antibody configured to bind the analyte of interest in the sample; binding the analyte of interest with the capture antibody; separating the analyte of interest from the sample by applying a magnetic field to the analysis chamber to pull down the magnetic conjugates with analyte of interest associated therewith; contacting the sample with a reporter conjugate comprising a reporter and a reporter antibody configured to bind the analyte of interest in the sample; binding the analyte of interest with the reporter antibody; and detecting the presence, absence, or level of the analyte of interest by detecting the reporter with a light source and photodetector.

In some embodiments, the methods described herein may be performed on a negative sample (i.e., a sample that does not include the analyte of interest) and thereby determine the absence of the analyte of interest in the sample.

In an embodiment, the methods described herein may include the steps of adding sample to an analysis chamber; contacting the sample with a magnetic conjugate comprising a magnetic particle and a capture antibody configured to bind the analyte of interest in the sample; binding the analyte of interest with the capture antibody; contacting the sample with a reporter-labeled analyte configured to bind the magnetic conjugate in the absence of the analyte of interest in the sample; separating the analyte of interest from the sample by applying a magnetic field to the analysis chamber; and detecting the presence, absence, or level of the analyte of interest by detecting the reporter.

In an embodiment, the methods described herein may include the steps of adding sample to an analysis chamber; contacting the sample with a reporter conjugate comprising a reporter and a reporter antibody configured to bind the analyte of interest in the sample; binding the analyte of interest with the reporter antibody; contacting the sample with a magnetic particle-labeled analyte configured to bind the reporter conjugate in the absence of the analyte of interest in the sample; separating the magnetic particle-labeled analyte from the sample by applying a magnetic field to the sample; and detecting the presence, absence, or level of the analyte of interest by detecting the reporter.

In an embodiment, the methods described herein may include the steps of adding sample to an analysis chamber; contacting the sample with a magnetic conjugate comprising a magnetic particle and a capture antibody configured to bind the analyte of interest in the sample; binding the analyte of interest with the capture antibody; contacting the sample with a reporter antibody comprising a biotin label configured to bind the analyte of interest in the sample; contacting the sample with a reporter comprising a streptavidin label configured to bind the biotin label; separating the analyte of interest from the sample by applying a magnetic field to the sample; and detecting the presence, absence, or level of the analyte of interest by detecting the reporter.

In some embodiments, where the methods described herein include a contacting step (e.g., contacting the sample with a magnetic conjugate, a reporter antibody, a reporter-labeled conjugate, and/or a reporter conjugate), such contacting step may include incubating the sample, which may contain an analyte, with the respective magnetic conjugate, reporter antibody, reporter-labeled conjugate, and/or reporter conjugate for a selected period of time and a selected temperature.

### BRIEF DESCRIPTION OF THE DRAWINGS

The foregoing summary, as well as the following detailed description of the invention, will be better understood when read in conjunction with the appended drawings.

FIGS. 1A to 1D illustrate exemplary features of a sandwich mode for an immunoassay described herein where antigen is present or absent. FIG. 1A illustrates exemplary parameters for the sandwich mode: (a) analyte of interest (e.g., hCG); (b) Antibody #1 that is specific to the analyte of interest (e.g., MoLogic Anti-hCG 4F9); (c) Antibody #2 that is specific to the analyte of interest (e.g., Medix Anti-hCG 5011); (d) Antibodies 1 and 2 are able to simultaneously bind to the analyte as shown here; (e) fluorescent reporter; and (f) magnetic particle. In the presence of analyte, a complex will form that is both labeled and can be attracted to a magnet. FIG. 1B illustrates the addition of magnetic conjugate and reporter conjugate. FIG. 1C illustrates the magnetic pulldown. FIG. 1D illustrates the detection of the analyte.

FIGS. 2A to 2F illustrate exemplary features of a separate addition mode for an immunoassay described herein where antigen is present or absent. FIG. 2A illustrates exemplary parameters for the separate addition mode: (a) analyte of interest (hCG); (b) Antibody #1 that is specific to the analyte of interest (e.g., MoLogic Anti-hCG 4F9); (c) Antibody #2 that is specific to the analyte of interest (e.g., Medix Anti-hCG 5011); (d) Antibodies 1 and 2 are able to simultaneously bind to the analyte as shown here; (e) fluorescent reporter; and (f) magnetic particle. FIG. 2B illustrates the addition of magnetic conjugate. FIG. 2C illustrates a magnetic pulldown. FIG. 2D illustrates the addition of reporter conjugate. FIG. 2E illustrates a second magnetic pulldown. FIG. 2F illustrates the detection of the analyte.

FIGS. 3A to 3E illustrate exemplary features of a competitive mode for an immunoassay described herein where antigen is present or absent. FIG. 3A illustrates exemplary parameters for the competitive mode: (a) Analyte of interest; (b) Antibody that is specific to the analyte of interest; (c) fluorescent reporter; and (d) magnetic particle. In the absence of analyte, the binding site on the antibody remains available, thus allowing the formulation of a complex between a magnetic particle and a reporter. In the presence of analyte, a free antigen can block the binding site on the antibody, thus preventing formation of a complex with the magnet particle and a reporter. FIG. 3B illustrates the addition of magnetic conjugate. FIG. 3C illustrates the addition of reporter-labeled analyte. FIG. 3D illustrates a magnetic pulldown. FIG. 3E illustrates the detection of the analyte.

FIG. 4A to 4G illustrate exemplary features of a tertiary mode for an immunoassay described herein where antigen is present or absent. FIG. 4A illustrates exemplary parameters for the tertiary mode: (a) Analyte of interest; (b) Antibody #1 that is specific to the analyte of interest and labeled with a biotin; (c) Antibody #2 that is specific to the analyte of interest; (d) Antibodies 1 and 2 are able to simultaneously bind to the analyte as shown here; (e) fluorescent reporter functionalized with streptavidin; and (f) magnetic particle. In the presence of analyte, a complex will form with a magnetic particle labeled biotin. When the streptavidin-labeled reporter is added to this complex, the reporter will bind to the complex and will result in a fluorescent complex that will be attracted to a magnet. FIG. 4B illustrates the addition of magnetic conjugate. FIG. 4C illustrates a magnetic pulldown. FIG. 4D illustrates the addition of a biotin labeled antibody. FIG. 4E illustrates the addition of streptavidin labeled reporter. FIG. 4F illustrates a second magnetic pulldown. FIG. 4G illustrates the detection of the analyte.

FIG. 5 illustrates the sensitivity of an assay of the invention for detecting Human Chorionic Gonadotropin (hCG).*Elecsys Electrochemiluminescence immunoassay HCG STAT (cobas e 601 module, cobas e 602 module). 1 fM=10⁻¹⁵ moles/liter.

FIG. 6 illustrates the sensitivity of an assay of the invention for detecting Luteinizing Hormone (LH). *Elecsys Electrochemiluminescence immunoassay LH (cobas). 1 fM=10⁻¹⁵ moles/liter.

FIG. 7 illustrates the sensitivity of an assay of the invention for detecting Prostate Specific Antigen (PSA). *Elecsys Electrochemiluminescence immunoassay free PSA (cobas). 1 fM=10⁻¹⁵ moles/liter.

FIGS. 8A to 8D illustrate exemplary features of a bacteria detecting mode for an immunoassay described herein where bacteria is present or absent. FIG. 8A illustrates exemplary parameter for the bacteria detecting mode: (a) Bacterium of interest; (b) Antibody #1 that is specific to an analyte on the surface of the bacterium; (c) Antibody #2 (may be the same as Antibody #1) that is specific to an analyte on the surface of the bacterium; (d) fluorescent reporter; and (e) magnetic particle. Many labeled antibodies can bind to a single bacterium at once resulting in a bacterium that can be attracted to a magnet and is fluorescently labeled. FIG. 8B illustrates the addition of magnetic conjugate and reporter conjugate. FIG. 8C illustrates a magnetic pulldown. FIG. 8D illustrates the detection of the analyte.

FIG. 9 illustrates the sensitivity of an assay of the invention for detecting Group A Strep.

FIG. 10 illustrates the detection of E3G in an exemplary competitive mode immunoassay of the invention.

FIG. 11 illustrates a correlation between measurements of PSA for an immunoassay described herein compared to those performed in a clinical laboratory.

FIG. 12 illustrates a typical test of hCG in urine samples as measured by an immunoassay described herein.

FIG. 13 illustrates a plot quantifying the results from FIG. 12.

FIG. 14 illustrates a stair-step curve showing the fraction positive as a function of days post ovulation. An immunoassay as described herein (i.e., Confer Magneto) is shown in purple. First Response is shown in pink. For reference in black and white, at day 10, Confer Magneto is the top curve and First Response is the bottom curve.

FIG. 15 illustrates a summary of the pregnancy data showing the percent of samples that read positive for a given number of days before First Response.

FIG. 16 illustrates a cis excitation method using a dichroic mirror arrangement with a 405 nm laser excitation source. Dichroic Mirror: Thor Labs, DMLP490R, 25 mm×36 mm Longpass Dichroic Mirror, 490 nm Cutoff. Filter: Thor Labs FGL610 Filter.

FIGS. 17A and 17B illustrate the results of a protocol for analyzing serum samples to detect C-reactive protein. FIG. 17A illustrates a CRP concentration series in buffer and FIG. 17B illustrates a CRP concentration series in spiked serium.

### DETAILED DESCRIPTION OF THE INVENTION

The invention is based, in part, on the discovery that analytes of interest, e.g. for health-related applications, can be detected in a sensitive and efficient manner using magnetic separation.

In some aspects, the invention provides a method for detecting the presence, absence or level of an analyte in a solution, comprising placing a labeled binding agent under conditions that allow for binding of the binding agent to the analyte; placing a particle comprising a binding agent under conditions that allow for binding of the binding agent to the analyte; applying a magnetic field with sufficient strength to separate resultant complexes comprising the analyte and label from the solution; and detecting the label in the complexes.

In various embodiments, the amount of detected label is compared in the presence or absence of the magnetic field. For instance, in some embodiments, the detection of the label (e.g. using any of the techniques described herein) is indicative of the presence of the analyte. Further, in some embodiments, the detection of the amount of label (e.g. using any of the techniques described herein) is indicative of the amount of analyte. In various embodiments, the amount of detected label is proportional to the amount of analyte. In some embodiments, no (or minimal) detection of the label (e.g. using any of the techniques described herein) is indicative of the absence (or substantial absence) of the analyte.

In various embodiments, the information regarding presence, absence, or level of an analyte of interest in a sample directs a healthcare or health-related lifestyle decision.

### Immunoassay

The invention described herein is directed, in part, to improved immunoassays for detecting analytes for detecting analytes of interest in samples, which may include samples of bodily fluids.

In an embodiment, the invention includes immunoassays that may be used to detect the presence, absence, or level of an analyte of interest in a sample.

In some embodiments, the immunoassays described herein may include an analysis chamber that may contain a sample, a magnetic conjugate, a reporter or a reporter conjugate, a magnet, a light source, and/or a photodetector.

In some embodiments, the immunoassays described herein may include a sample collector associated with, or otherwise in fluid communication with, the analysis chamber. In some embodiments, the sample collector may include as an absorbent and/or wicking material that may absorb the sample and then feed the sample into the analysis chamber. In some embodiments, one or more of the magnetic conjugate, reporter, and reporter conjugate may be disposed at the sample collector such that when a sample is added to the sample collector, the sample may contact the magnetic conjugate, reporter, reporter conjugate, or a combination thereof. In some embodiments, one or more of the magnetic conjugate, reporter, and reporter conjugate may be imbedded in a portion of the sample collector.

In some embodiments, the sample collector includes a sponge, foam, or membrane, such as a polyurethane sponge or foam, or another adsorbent and/or absorbent material. In some embodiments, the sample collector may include a cellulose, nitro-cellulose, and/or polyvinyl difluoride (PVDF) membrane, sponge, or foam. In some embodiments, the sample collector may be a Porex adsorbent, which may be PE/PET based. In some embodiments, the Porex adsorbent may include Porex conjugate release layer that may be sintered PE based.

In some embodiments, the magnet may be a permanent magnet that may be separated from the analysis chamber in order to apply a magnetic field to the analysis chamber. In some embodiments, the magnet may be an electromagnet that may be activated or deactivated in order to apply a magnetic field to the analysis chamber.

In some embodiments, the light source connected to the analysis chamber and may be configured to transmit light through a portion of the analysis chamber.

In some embodiments, the analysis chamber may be one chamber, or two chambers, or three chambers, or four chambers. In some embodiments, the analysis chamber may be one or more chambers, or two or more chambers, or three or more chambers, or four or more chambers. In some embodiments, the analysis chamber may include a plurality of chambers. In some embodiments, the plurality of chambers may be in fluid communication. In some embodiments, the sample, reporter or reporter conjugate, and magnetic conjugate may be mixed in a first chamber. In certain embodiments, the magnetic field may be applied in a second chamber and the light source connected to the analysis chamber may be configured to transmit light through the second chamber. In some embodiments, the method steps described herein may each be performed in separate chambers of the analysis chamber. In some embodiments, the analysis chamber may be one chamber and all method steps may be performed in the same chamber.

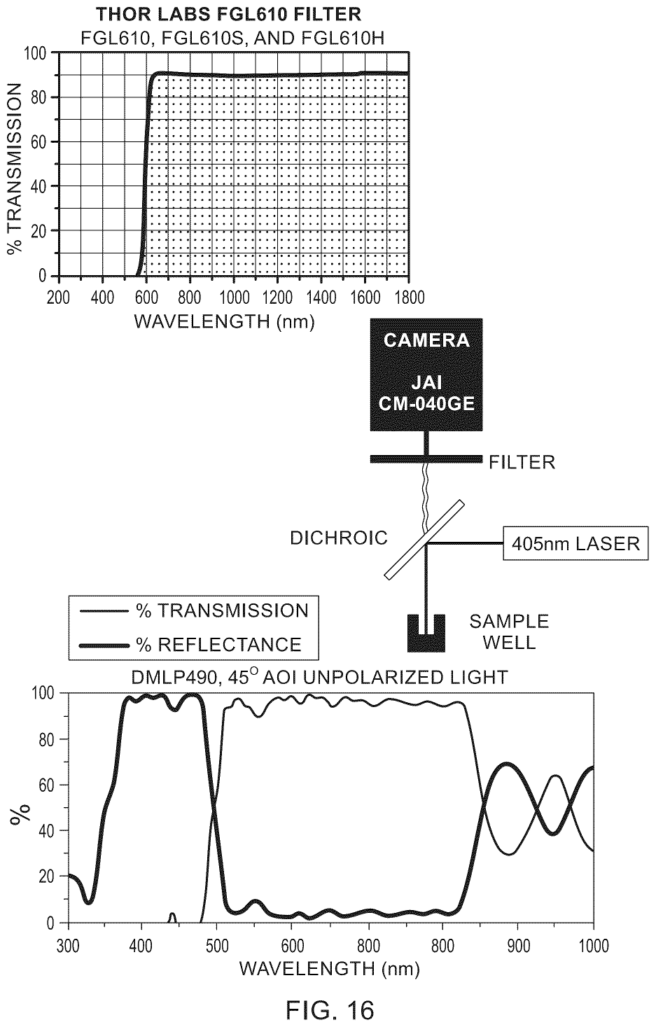

In some embodiments, a photodetector may be connected to the analysis chamber (e.g., facing, in line with, or opposite the light source) and may be configured to detect light transmitted through the analysis chamber by the light source and thereby measure transmittance and/or absorbance of the light. In some embodiments, the photodetector may be connected to the analysis chamber, orthogonal to the light source (orthogonal illumination), and may be configured to detect fluorescence and/or phosphorescence of a reporter or reporter conjugate in a portion of the analysis chamber. In some embodiments, the photodetector may be connected to the analysis chamber, opposite to the light source (trans illumination), and may be configured to detect fluorescence and/or phosphorescence of a reporter or reporter conjugate in a portion of the analysis chamber. In some embodiments, the photodetector may be connected to the analysis chamber, in line with the light source (e.g., by way of a dichroic mirror as shown in FIG. 16 (cis illumination)), and may be configured to detect fluorescence and/or phosphorescence of a reporter or reporter conjugate in a portion of the analysis chamber. In some embodiments, a photodetector may include one or more photomultiplier tube detectors and photodiode detectors. As used herein, the term “photomultiplier” or “photomultiplier tube” refers to optical detection components that convert incident photons into electrons via the photoelectric effect and secondary electron emission. The term photomultiplier tube is meant to include devices that contain separate dynodes for current multiplication as well as those devices that contain one or more channel electron multipliers. As used herein, the term “optical detector” or “photodetector” refers to a device that generates an output signal when irradiated with optical energy. Thus, in its broadest sense the term optical detector system is taken to mean a device for converting energy from one form to another for the purpose of measurement of a physical quantity or for information transfer. Optical detectors include but are not limited to photomultipliers and photodiodes. As used herein, the term “photodiode” refers to a solid-state light detector type including, but not limited to PN, PIN, APD, CMOS, and CCD. In some embodiments, the photodetector may include one or more of a PN based detector, a PIN based detector, an APD based detector, a CMOS based detector, and a CCD based detector. In some embodiments, the analysis chamber comprises a photodetector as described herein. In some embodiments, the analysis chamber comprises one or more of a PN based detector, a PIN based detector, an APD based detector, a CMOS based detector, and a CCD based detector.

In some embodiments, the magnetic conjugate described herein may include a magnetic particle and a capture antibody associated therewith. In some embodiments, the magnetic particle may be bound to the capture antibody.

In some embodiments, the reporter conjugate described herein may include a reporter and a reporter antibody. In some embodiments, the reporter may be bound to the reporter antibody.

### Binding Partners and Antibodies

In some embodiments, the capture moiety and the reporter binding moiety may be the same or different. In some embodiments, the capture moiety and/or the reporter binding moiety may be an antibody, an antigen-binding fragment, an antigen, a receptor, a ligand, an aptamer, an aptamer receptor, a nucleic acid, or a small molecule. In some embodiments, the capture moiety may be a capture antibody. In some embodiments, the reporter binding moiety may be a reporter antibody.

In some embodiments, the capture antibody and/or the reporter antibody may be selected from the group consisting of antibodies for the analytes of interest described herein. In some embodiments, the capture antibody and/or the reporter antibody may be selected from the group consisting of an anti-hCG antibody, anti-LH antibody, anti-PSA antibody, anti-HSV antibody, anti-E3G antibody, an anti-bacterial cell surface protein antibody, an anti-hemoglobin AlC antibody, an anti-C-reactive protein antibody, an anti-inflammation biomarker antibody, an anti-troponin antibody, an anti-lyme disease antibody, an anti-LDL biomarker antibody, an anti-HDL biomarker antibody, an anti-total cholesterol biomarker antibody, an anti-estrogen antibody, an anti-progesterone antibody, an anti-thyroid stimulation hormone antibody, an anti-hepatitis C virus biomarker antibody, an anti-rhino virus biomarker antibody, an anti-influenza biomarker antibody, an anti-liver function biomarker antibody, an anti-fertility biomarker antibody, an anti-muscle fatigue biomarker antibody, and combinations thereof.

In some embodiments, the capture antibody and/or the reporter antibody may be an antibody that may bind an analyte of interest selected from the group consisting of human chorionic gonadotropin (hCG), luteinizing hormone (LH)/Lutropin, prostate specific antigen (PSA), herpes simplex virus (HSV) antibodies, estrone-3-glucuronide (E3G), bacteria, hemoglobin AlC, C-reactive protein, an inflammation biomarker, troponin, lyme disease antigen, lyme disease antibodies, an LDL biomarker, an HDL biomarker, a total cholesterol biomarker, thyroid stimulating hormone, a hepatitis C virus biomarker, a rhino virus biomarker, an influenza virus biomarker, a liver function biomarker, estrogen, progesterone, lactic acid, and combinations thereof. In some embodiments, the bacteria may be Streptococcus-A, Chlamydia, and/or Gonorrhea. In some embodiments, the inflammation biomarker may be CRP, SAA, and/or MP8. In some embodiments, the liver function biomarker may be ALT and/or AST. In some embodiments, the analyte of interest may be selected from the group consisting of an ovulation biomarker, a pregnancy biomarker, a strep throat biomarker, a prostate cancer biomarker, a herpes biomarker, a diabetes biomarker, an inflammation biomarker, a heart attack biomarker, a chlamydia biomarker, a bacteria biomarker, a lyme disease biomarker, a cholesterol biomarker, a hypothydroidism biomarker, a hepatitis C biomarker, a rhino virus biomarker, an influenza biomarker, a liver function biomarker, a fertility biomarker, a muscle fatigue biomarker, and combinations thereof.

In various embodiments, the binding partners described herein may include, without limitation, antibodies including but not limited to single chain antibodies, antigen-binding antibody fragments, antigens (to be used to bind to their antibodies, for example), receptors, ligands, aptamers, aptamer receptors, nucleic acids, small molecules, and the like.

In some embodiments, the capture antibody and/or the reporter antibody may be selected from the group consisting of INN-hCG-2, INN-hCG-2, 5008-SP5, 5008-SP5, and 5011 SPRN-1, or functional variants thereof. In some embodiments, the capture antibody and/or the reporter antibody may be one or more antibodies described in Table 1.

Listed in Table 1 are exemplary antibody pairs that can be used to detect hCG (e.g., selecting one or two of the following to make a pair):

TABLE 1

| ISOBMii | | |
|---|---|---|
| Ab Codes | Owner | Owner Codes |
| 382 | Stenman | F16-6G5 |
| 383 | Medix | 5501 SP-1 |
| 384 | Stenman | F52-3F8 |
| 385 | Medix | 5503 SPI |
| 386 | Stenman | F94-8F8 |
| 387 | Medix | 5009 SP-5 |
| 388 | Medix | 5006 SP-5 |
| 389 | Stenman | F140-11C5 |
| 390 | Medix | 5008 SP-5 |
| 391 | Medix | 6601 SPR-5 |
| 392 | Stenman | F20-6E11 |
| 393 | Stenman | F52-3C11 |
| 394 | Medix | 5014 SPTN-5 |
| 395 | Abbott | 71752 |
| 396 | Stenman | F132-3C10 |
| 397 | Stenman | F142-7F3 |
| 398 | Stenman | F26-2G11 |
| 399 | Stenman | F95-5C4 |
| 400 | Abbott | 95658 |
| 401 | Stenman | F95-1E8 |
| 402 | Medix | 5004 SP-1 |
| 403 | Roche | M-INN2 |
| 404 | Stenman | F26-7E10 |
| 405 | Stenman | F95-1B2 |
| 406 | Medix | 5011 SPRN-1 |
| 407 | Stenman | F19-9C11 |
| 408 | Medix | 5016 SPRN-5 |
| 409 | Medix | 5012 SPRN-1 |
| 410 | Roche | M-BCG005 |
| 411 | Roche | M-1F7.9 |
| 412 | Siemens | 34/25.2.2 |
| 413 | Mologic | D101 |
| 414 | Paus | E26 |
| 415 | Siemens | 3A11 |
| 416 | Paus | E30 |
| 417 | Roche | M-INN22 |
| 418 | Siemens | 2F11 |
| 419 | Paus | E27 |
| 420 | Roche | M-94.139 |
| 421 | Siemens | 411/100.1.1.200.4.2 |
| 422 | Paus | E28 |
| 423 | Mologic | D102 |
| 424 | Siemens | 1G4 |
| 425 | Siemens | 500000 |
| 426 | Siemens | 16 E 2 |
| 427 | Siemens | 34A8.1.1 |
| 432 | Medix | 41-3-9 |
| 433 | Medix | 45A10 |
| 428 sheep | Mologic | 8F11 sheep |
| 429 sheep | Mologic | 9F10 sheep |
| 430 sheep | Mologic | 8G5 sheep |
| 431 sheep | Mologic | 618 sheep poly |
| 434a | INN | hCG111 |
| 435a | INN | hCG2 |
| 436a | INN | hCG40 |
| 437a | INN | hCG64 |
| 438a | INN | hCG53 |
| 439a | INN | hCG68 |
| 440a | INN | hCG26 |
| 441a | INN | bLH1 |
| 442a | INN | hCG58 |
| 443a | INN | hCG112 |
| 444a | INN | hCG106 |
| 445a | INN | hCG24 |
| 446a | INN | hCG45 |
| 447a | INN | hCG10 |
| 448a | INN | hCG103 |
| 449a | INN | hCG22 |
| 450a | Stahli | h54 |

Several important factors have been identified for choosing antibodies pairs which work successfully in urine. For example, the ability to simultaneously bind is a prerequisite. In some embodiments, the antibodies must be able to bind to fully intact hCG as well as the beta subunit. Antibodies which have high affinities, but low on-rates, work but require long incubations. The following antibodies have been successfully used to detect endogenous hCG in humane urine: INN-hCG-2, INN-hCG-22, 5008-SP5, 5014-SPTN5, and 5011 SPRN-1. Accordingly, an embodiment of the invention pertains to the use or one, or two of INN-hCG-2, INN-hCG-22, 5008-SP5, 5014-SPTN5, and 5011 SPRN-1, or functional fragments thereof.

In some embodiments, the capture antibody and/or the reporter antibody may be Fitzgerald 10-L15A and 10-L15B, or functional variants thereof.

In some embodiments, the capture antibody and/or the reporter antibody may be anti-PSA 5001 (Medix), anti-PsA 5012 (Medix), or functional variants thereof.

In some embodiments, the capture antibody and/or the reporter antibody may be polyclonal antibodies targeting the Strep-A antigen or monoclonal Strep-A 2601 SPTN-5 or 2603 SPTN-5 antibodies, or functional variants thereof manufactured by Biospacific.

In various embodiments, the analyte to be detected may be virtually any analyte provided that binding partners specific for the analyte are available. In various embodiments, the analyte can be bound by at least two binding partners simultaneously. In various embodiments, the analyte is bound by the binding partners at the same epitope or at different epitopes. In various embodiments, the analytes may be or may comprise nucleic acids, peptides or proteins, carbohydrates, lipids, or any combination thereof.

In various embodiments, the invention described herein contemplates the detection of Human chorionic gonadotropin (hCG), for example, as part of a pregnancy test. Fully intact hCG includes a dimer formed between two hCG subunits, alpha-hCG and beta-hCG. In some embodiments, the hCG is detected using antibodies, for example, a pair of antibodies that recognize one or more epitopes on alpha-hCG and beta-hCG. In an embodiment, the hCG is detected using antibodies that recognize beta-hCG. In various embodiments, any known antibodies directed against alpha-hCG or beta-hCG may be utilized in the invention described herein. In some embodiments, the antibodies include INN-hCG-2, INN-hCG-2, 5008-SP5, 5008-SP5, and 5011 SPRN-1, or functional variants thereof. In some embodiments, the methods described herein can detect hCG earlier and with greater accuracy than conventional pregnancy tests on the market such as those pregnancy tests developed by First Response (as used herein, “First Response” refers to an over the counter chromatographic immunoassay for the qualitative detection of human chorionic gonadotropin (hCG)).

In various embodiments, the invention described herein contemplates the detection of luteinizing hormone (LH)/Lutropin, for example, as part of a test for identifying ovulation. Exemplary antibodies that recognize LH that may be used in methods described herein include, but are not limited to, Fitzgerald 10-L15A and 10-L15B, or functional variants thereof.

In some embodiments, the invention described herein further contemplates the detection of estrone-3-glucuronide (E3G) as another biomarker for identifying ovulation. In some embodiments, the methods described herein can detect LH or E3G earlier and with greater accuracy than conventional ovulation tests on the market such as the ClearBlue Digital Ovulation Test (an over the counter LH test) or other ovulation tests developed by ClearBlue. In some embodiments, the methods described herein are particularly suited for predicting ovulation in women with polycystic ovary syndrome (PCOS) who cannot use the ovulation tests currently on the market due to their high LH baseline.

In various embodiments, the invention contemplates the detection of Prostate Specific Antigen (PSA). Exemplary antibodies that recognize PSA that may be used in methods described herein include, but are not limited to, anti-PSA 5001 (Medix), anti-PsA 5012 (Medix), or functional variants thereof.

In various embodiments, the invention contemplates the detection of Herpes Simplex Virus (HSV) or antibodies against HSV present in the blood or serum. In some embodiments, the methods described herein relate to the detection of HSV-1 (oral herpes) or antibodies against HSV-1 present in the blood or serum. In other embodiments, the methods described herein relate to the detection of HSV-2 (genital herpes) or antibodies against HSV-2. For example, an HSV-1 or HSV-2 antigen may be used as binding partners to detect the presence of antibodies against HSV-1 or HSV-2 in the blood or serum.

In various embodiments, the invention contemplates the detection of Streptococcus-A (Strep-A). Exemplary antibodies that may be utilized for the detection of Strep-A include, but are not limited to, polyclonal antibodies targeting the Strep-A antigen or monoclonal Strep-A 2601 SPTN-5 or 2603 SPTN-5 antibodies, or functional variants thereof manufactured by Biospacific. In some embodiments, the methods described herein can detect Strep-A antigen earlier and with greater accuracy than conventional tests such as the QuickVue Dipstick Strep A test (an immunofluorescence test to detect Group A Streptococcal antigens from throat swabs of symptomatic patient).

In an embodiment, the methods described herein are at least about 10 times, about 20 times, about 30 times, about 40 times, about 50 times, about 60 times, about 70 times, about 80 times, about 90 times, or at least about 100 times more sensitive than other rapid tests currently on the market, e.g., the QuickVue Dipstick Strep A test. In various embodiments, the methods described herein have enhanced specificity to Strep-A compared to other Streptococcus bacteria such as Strep-B, Strep-C or Strep-G.

In various embodiments, the invention contemplates the detection of various infections, including gonorrhea and chlamydia. Exemplary antigens that may be detected using antibodies in the methods described herein include, but are not limited to, chlamydial LPS KDO-trisaccharide, chlamydial major outer membrane protein, all antigens of Neisseria gonorrhea including any major outer membrane protein.

In various embodiments, the invention contemplates the detection of various diseases or conditions including diabetes and inflammation. Exemplary antigens that may be detected using antibodies in the methods described herein include, but are not limited to, Hemoglobin AlC and C-reactive protein. Additional antigens that may be detected by methods described herein include any known antigen that may be detected by ELISA or sandwich ELISA immunoassays currently on the market.

In certain embodiments, the methods described herein may encompass the use of one antibody (e.g., a capture antibody or a reporter antibody). In various embodiments, the invention described herein contemplates the use of multiple antibodies, such as, for example, one or more capture antibodies and one or more reporter antibodies. In some embodiments, the invention described herein may utilize at least about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 250, about 300, about 350, about 400, about 450, or about 500, about 750, about 1000, about 1250, about 1500, about 1750, about 2000, about 3000, about 4000, about 5000, about 6000, about 7000, about 8000, about 9000, about 10000 antibodies. In embodiments, the invention described herein may utilize at least about 2, about 3, about 4, about 5, about 6, about 7, about 8, about 9, about 10, about 11, about 12, about 13, about 14, about 15, about 16, about 17, about 18, about 19, about 20, about 25, about 30, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, or about 250, or about 500, or about 750, or about 1000, or about 1250, or about 1500, or about 1750, or about 2000, or about 3000, or about 4000, or about 5000, or about 6000, or about 7000, or about 8000, or about 9000, or about 10000 antibody pairs.

In some embodiments, the invention described herein may utilize multivalent antibodies. For example, the invention may involve coupling of bi-valent or trivalent single-chain variable fragment antibodies (e.g., each of which can contain about 4 or about 6 analyte, or more, binding sites, respectively). In other embodiments, methods described herein may involve chemically forming aggregates of multiple antibodies. This could be performed with a variety of multifunctional linkers.

It is contemplated that use of multiple binding partners may allow for improvements in the speed and sensitivity of analyte detection.

In various embodiments, methods described herein minimize false positive signals. In some embodiments, the present methods reduce signals by controlling solution pH. In some embodiments, the solution pH is controlled by the use of appropriate buffers, which can be specific for the antibodies used. In some embodiments, Tris/Borate/EDTA buffer and/or buffers with EDTA are utilized. Various buffers may be utilized in the invention. Exemplary buffers that may be utilized for running gels in the invention include, but are not limited to, single buffers systems such as Sodium Borate, Sodium Acetate, Sodium Citrate, Lithium Borate, Tris/Acetic Acid/EDTA, Tris/Acetic Acid, Tris-Acetate, Tris Acetate EDTA, Tris/TAPS/EDTA Buffer, Bis-Tris/HCl buffer, Tris-Acetate SDS, MOPS, MOPS/Tris/SDS/EDTA, MOPS/Tris/EDTA, MOPS/Tris/SDS, MOPS/Tris, MES, MES/Tris/SDS/EDTA, MES/Tris/EDTA, MES/Tris/SDS, MES/Tris, Tris-glycine, or dual buffer systems such as Tris EDTA on one side and Boric Acid on the other side of the gel. Additional exemplary buffers that may be utilized for stabilizing pH include, but are not limited to, Sodium Borate, Sodium Acetate, Sodium Citrate, Lithium Borate, Tris-HCl, TAPS, Tris/Acetic Acid/EDTA, Tris-Acetate, Tris Acetate EDTA, Tris/TAPS/EDTA Buffer, Ammonium Bicarbonate, Sodium Bicarbonate, Phosphate buffer, Guanidine Hydrochloride, Guanidine Thiocyanate, Bis-Tris/HCl buffer, Tris-Acetate SDS, MOPS, MOPS/Tris/EDTA, MOPS/Tris/SDS, MOPS/Tris, MES, MES/Tris/SDS/EDTA, MES/Tris/SDS, MES/Tris, and Tris-glycine.

In some embodiments, passivating agents such as Tween, BSA, poly ethylene glycol, or casein are used. Additional exemplary passivating agents that may be utilized in the invention include, but are not limited to, Glycerol, Sucrose, Glucose, TritonX, SDS, LDS, Sigmacoat, DNA oligos, Fish Gelatin, Whole sera, Polyvinyl alcohol, polyvinylpyrrolidone, salmon-sperm DNA, Silanes, and Silica.

### Methods of Using Immunoassays

Methods described herein include methods for detecting the presence, absence, or level of an analyte of interest in a sample.

In some embodiments, the methods described herein may include the steps of:
a. contacting the sample with a magnetic conjugate comprising a magnetic particle and a capture antibody configured to bind the analyte of interest in the sample;
b. contacting the sample with a reporter conjugate comprising a reporter and a reporter antibody configured to bind the analyte of interest in the sample;
c. binding the analyte of interest with the capture antibody and the reporter antibody;
d. separating the analyte of interest from the sample by applying a magnetic field to the analysis chamber to pull down the magnetic conjugates with analyte of interest associated therewith; and
e. detecting the presence, absence, or level of the analyte of interest by detecting the reporter with a light source and photodetector.

In some embodiments, the methods described herein may include the steps of:
a. contacting the sample with a reporter conjugate comprising a reporter and a reporter antibody configured to bind the analyte of interest in the sample;
b. contacting the sample with a magnetic conjugate comprising a magnetic particle and a capture antibody configured to bind the analyte of interest in the sample;
c. binding the analyte of interest with the capture antibody and the reporter antibody;
d. separating the analyte of interest from the sample by applying a magnetic field to the analysis chamber to pull down the magnetic conjugates with analyte of interest associated therewith; and
e. detecting the presence, absence, or level of the analyte of interest by detecting the reporter with a light source and photodetector.

In some embodiments, the methods described herein may include the steps of:
a. contacting a sample with a magnetic conjugate comprising a magnetic particle and a capture antibody configured to bind the analyte of interest in the sample;
b. binding the analyte of interest with the capture antibody;
c. separating the analyte of interest from the sample by applying a magnetic field to the analysis chamber to pull down the magnetic conjugates with analyte of interest associated there with;
d. contacting the sample with a reporter conjugate comprising a reporter and a reporter antibody configured to bind the analyte of interest in the sample;
e. binding the analyte of interest with the reporter antibody;
f. separating the analyte of interest from the sample by applying a magnetic field to the analysis chamber to pull down the magnetic conjugates with analyte of interest and the reporter conjugate associated therewith; and
g. detecting the presence, absence, or level of the analyte of interest by detecting the reporter with a light source and photodetector.

In some embodiments, the methods described herein may include the steps of:
a. contacting a sample with a magnetic conjugate comprising a magnetic particle and a capture antibody configured to bind the analyte of interest in the sample;
b. binding the analyte of interest with the capture antibody;
c. contacting the sample with a reporter-labeled analyte configured to bind the magnetic conjugate in the absence of the analyte of interest in the sample;
d. separating the analyte of interest from the sample by applying a magnetic field to the analysis chamber to pull down the magnetic conjugates with analyte of interest associated there with; and
e. detecting the presence, absence, or level of the analyte of interest by detecting the reporter with a light source and photodetector.

In some embodiments, the methods described herein may include the steps of:
a. contacting a sample with a reporter conjugate comprising a reporter and a reporter antibody configured to bind the analyte of interest in the sample;
b. binding the analyte of interest with the reporter antibody;
c. contacting the sample with a magnetic particle-labeled analyte configured to bind the reporter conjugate in the absence of the analyte of interest in the sample;
d. separating the magnetic particle-labeled analyte from the sample by applying a magnetic field to the sample; and
e. detecting the presence, absence, or level of the analyte of interest by detecting the reporter with a light source and photodetector.

In some embodiments, the methods described herein may include the steps of:
a. contacting a sample with a magnetic conjugate comprising a magnetic particle and a capture antibody configured to bind the analyte of interest in the sample;
b. binding the analyte of interest with the capture antibody;
c. contacting the sample with a reporter antibody comprising a biotin label configured to bind the analyte of interest in the sample;
d. contacting the sample with a reporter comprising a streptavidin label configured to bind the biotin label;
e. separating the analyte of interest from the sample by applying a magnetic field to the sample; and
f. detecting the presence, absence, or level of the analyte of interest by detecting the reporter.

In some embodiments of the methods described herein, the magnetic conjugate (including a capture antibody) may be added simultaneously with a reporter or reporter conjugate.

In some embodiments of the methods described herein, the magnetic conjugate (including a capture antibody) and a reporter or reporter conjugate may be added separately.

In various embodiments, the methods of invention may include the step of adding sample to an analysis chamber. In some embodiments, adding sample to the analysis chamber may include delivering the sample to a sample collector (e.g., an absorbent and/or wicking material) in fluid communication with the analysis chamber. In some embodiments, the sample collector may then feed the sample into the analysis chamber.

In some embodiments, one or more of the magnetic conjugate, reporter, and reporter conjugate may be disposed at the sample collector and the steps of contacting the sample with the magnetic conjugate, reporter, or reporter conjugate may occur at the sample collector. In some embodiments, one or more of the magnetic conjugate, reporter, and reporter conjugate may be imbedded in a portion of the sample collector before a sample is added to the sample collector. In some embodiments, the methods described herein may include contacting the sample in the analysis chamber with a magnetic conjugate, reporter, and/or reporter conjugate.

In some embodiments of the methods described herein, the reporter may be a fluorescent reporter, a phosphorescent reporter, or a colorimetric reporter such as a colored particle that may be configured to measure absorbance or scattering of light (or, for example, the presence/absence of a certain color by colorimetric analysis).

In some embodiments, the methods described herein may further include the step of concentrating the analyte of interest in the sample by applying a magnetic field to the analysis chamber after contacting the sample with the magnetic conjugate; and then reducing the volume of the sample in the analysis chamber. In some embodiments, the methods described herein may further include the step of deactivating the magnetic field before contacting the sample with the reporter conjugate.

In some embodiments, reducing the volume of the sample in the analysis chamber may be performed by, for example, syphoning of a fraction of the volume or by removing the entire sample and resuspending in a new lesser volume.

In some embodiments, the methods described herein may further include the steps of concentrating the analyte of interest in the sample by applying a magnetic field to the analysis chamber after contacting the sample with the magnetic conjugate; removing a volume of the sample from the analysis chamber; and adding a volume of buffer and/or an additional volume of the sample to the analysis chamber. In some embodiments, the methods described herein may include the step of deactivating the magnetic field before contacting the sample with the reporter conjugate.

In some embodiments, the methods described herein may include the step of adding a volume of buffer and/or additional volumes of sample to the analysis chamber.

In some embodiments, the methods described herein may include the step of removing volumes of sample from the analysis chamber after a pull down of the magnetic conjugate (i.e., application of a magnetic field) and before or after contacting the sample with a reporter or reporter conjugate.

In some embodiments of the methods described herein the reporter antibody is labeled with biotin and the reporter is functionalized with streptavidin. In some embodiments of the methods described herein the reporter antibody is functionalized with streptavidin and the reporter is labeled with biotin.

In some embodiments, where the methods described herein include a contacting step (e.g., contacting the sample with a magnetic conjugate, a reporter antibody, a reporter-labeled conjugate, and/or a reporter conjugate), such contacting step may include incubating the sample, which may contain an analyte, with the respective magnetic conjugate, reporter antibody, reporter-labeled conjugate, and/or reporter conjugate for a selected period of time and a selected temperature.

In some embodiments of the methods described herein, the analyte of interest may be any analyte of interest described herein. In some embodiments, the analyte of interest may be selected from the group consisting of human chorionic gonadotropin (hCG), luteinizing hormone (LH)/Lutropin, prostate specific antigen (PSA), herpes simplex virus (HSV) antibodies, estrone-3-glucuronide (E3G), bacteria, hemoglobin AlC, C-reactive protein, an inflammation biomarker, troponin, lyme disease antigen, lyme disease antibodies, an LDL biomarker, an HDL biomarker, a total cholesterol biomarker, thyroid stimulating hormone, a hepatitis C virus biomarker, a rhino virus biomarker, an influenza virus biomarker, a liver function biomarker, estrogen, progesterone, lactic acid, and combinations thereof. In some embodiments, the bacteria may be Streptococcus-A, Chlamydia, and/or Gonorrhea. In some embodiments, the inflammation biomarker may be CRP, SAA, and/or MP8. In some embodiments, the liver function biomarker may be ALT and/or AST. In some embodiments, the analyte of interest may be selected from the group consisting of an ovulation biomarker, a pregnancy biomarker, a strep throat biomarker, a prostate cancer biomarker, a herpes biomarker, a diabetes biomarker, an inflammation biomarker, a heart attack biomarker, a chlamydia biomarker, a bacteria biomarker, a lyme disease biomarker, a cholesterol biomarker, a hypothydroidism biomarker, a hepatitis C biomarker, a rhino virus biomarker, an influenza biomarker, a liver function biomarker, a fertility biomarker, a muscle fatigue biomarker, and combinations thereof.

In some embodiments, an ovulation biomarker may be derived from a urine, blood, or serum based sample. In some embodiments, a pregnancy biomarker may be derived from a urine or blood based sample. In some embodiments, a strep throat biomarker may be derived from a saliva based sample. In some embodiments, a prostate cancer biomarker may be derived from a blood, serum, or urine based sample. In some embodiments, a herpes biomarker may be derived from a blood or saliva derived sample.

In certain embodiments, the analyte of interest may be selected from the group consisting of hCG, C-reactive protein, LH, PSA, HSV, E3G, a bacterium (e.g., Strep A), and combinations thereof.

In some embodiments of the methods described herein, the sample may be a bodily fluid as described herein. In some embodiments, the methods described herein may include obtaining a bodily fluid sample from a patient.

In certain embodiments, the methods described herein encompass a sandwich method, a separate addition method, a competitive method, and a tertiary method.

For example, the sandwich method described herein may be well suited for processing small fluid sample volumes in an immunoassay format. The separate addition method described herein may enable processing of larger fluid volumes with improved sensitivity. The competitive assay method may be useful for assaying in which a user cannot find both a capture antibody and a reporter antibody that may bind to the analyte simultaneously, e.g., where the analyte is a small molecule. The tertiary assay method may provide three binding events to enhance the kinetics of a system. The tertiary binding motif can be applied through a sandwich method, separate addition method, or competitive assay method formats.

In various embodiments, immunoassays, methods, and kits described herein allow for personal base lining for an analyte of interest. In such embodiments, the immunoassays, methods, and kits described herein allow for a determination of a normal analyte range for each individual user. In some embodiments, the user is alerted if there is any deviation from the individual's personal normal analyte range.

In various embodiments, the analyte (e.g., an antigen) that may be detected is any biomarker for a biological event. In some embodiments, the biological events may include a disease event (i.e., disease biomarker), an inflammation event (i.e., an inflammation biomarker), a reproduction event (i.e., a reproduction biomarker), and/or an aging event (i.e., an aging biomarker).

In various embodiments, the invention relates to the detection of a biomarker for a biological event using the systems and methods described herein. In various embodiments, there is provided a method of pregnancy detection using the systems and methods described herein. In various embodiments, there is provided a method of ovulation detection using the systems and methods described herein. In various embodiments, there is provided a method of prostate health detection (e.g. detecting the presence a cancer, or likelihood of developing the same) using the systems and methods described herein. In various embodiments, there is provided a method of herpes detection using the systems and methods described herein. In various embodiments, there is provided a method of streptococcal infection detection using the systems and methods described herein.

In various embodiments, the methods described herein include various detection techniques, e.g. for reporter signal. Such detection techniques may involve a microscope, a spectrophotometer, a fluorometer, a tube luminometer or plate luminometer, x-ray film, magnetic fields, a scintillator, a fluorescence activated cell sorting (FACS) apparatus, a microfluidics apparatus, a bead-based apparatus or the like.

In some embodiments, the magnetic particle is a paramagnetic particle. In some embodiments, the paramagnetic particle is a nanoparticle or a microparticle. In some embodiments, the paramagnetic particle is a bead, such as a nanobead or a microbead. The paramagnetic particle is, in various embodiments, a magnetic nano- or microbead, which allows the particle to be held and/or manipulated by magnets. In some embodiments the paramagnetic particle is a metallic nanoparticles coated with a thin (ca. 2 nm) graphene-like carbon layer. In some embodiments the paramagnetic particle is coated, e.g. streptavidin- or PEG-coated. Examples magnetic particles that can be used are DYNABEADs (THERMOFISHER), MACS beads (MILTENYI BIOTEC), TURBOBEADS (TURBOBEADS), ABSOLUTE MAG STREPTAVIDIN MAGNETIC PARTICLES (CREATIVE DIAGNOSTICS), and GOLD NANOPARTICLES (SIGMAALDRICH).

In some embodiments, the magnetic particles described herein may include a biocompatible coating that may be activated with amine groups or carboxyl groups to facilitate amid coupling. In some embodiments, the magnetic particles described herein may be activated with amine groups or carboxyl groups to facilitate amid coupling.

In some embodiments, the reporter particles described herein may include a biocompatible coating that may be activated with amine groups or carboxyl groups to facilitate amid coupling. In some embodiments, the reporter particles described herein may be activated with amine groups or carboxyl groups to facilitate amid coupling.

In some embodiments, the particles described herein may be be microparticles (e.g. microbeads), which are about 0.5 micrometers to about 500 micrometers in diameter (e.g. about 0.5 micrometers, or about 1 micrometer, or about 10 micrometers, or about 50 micrometers or about 100 micrometers or about 250 micrometers or about 500 micrometers).

In some embodiments, the particles described herein may be nanoparticles (e.g. nanobeads), which are smaller than 1 micrometer in diameter (e.g. about 5 to about 500 nanometers, e.g. about 5 nanometers, or about 10 nanometers, or about 50 nanometers, or about 100 nanometers, or about 250 nanometers, or about 500 nanometers). In some embodiments, the nanoparticles (e.g. nanobeads) have a mean particle diameter of 25-500 nm+/−5 nm, 25-500 nm+/−10 nm, 25-500 nm+/−15 nm, 25-500 nm+/−20 nm, 25-500 nm+/−25 nm, 25-500 nm+/−30 nm, 25-500 nm+/−35 nm, 25-500 nm+/−40 nm, 25-500 nm+/−45 nm, or 25-500 nm+/−50 nm. In some embodiments, the nanoparticles (e.g. nanobeads) have a mean particle diameter of about 20 to about 200 nm.

In some embodiments, the microparticles (e.g. microbeads) are about 0.5 micrometers to about 500 micrometers in diameter (e.g. about 0.5 micrometers, or about 1 micrometer, or about 10 micrometers, or about 50 micrometers or about 100 micrometers or about 250 micrometers or about 500 micrometers).

In some embodiments, the nanoparticles (e.g. nanobeads) are smaller than 1 micrometer in diameter (e.g. about 5 to about 500 nanometers, e.g. about 5 nanometers, or about 10 nanometers, or about 50 nanometers, or about 100 nanometers, or about 250 nanometers, or about 500 nanometers). In some embodiments, the nanoparticles (e.g. nanobeads) have a mean particle diameter of 25-500 nm+/−5 nm, 25-500 nm+/−10 nm, 25-500 nm+/−15 nm, 25-500 nm+/−20 nm, 25-500 nm+/−25 nm, 25-500 nm+/−30 nm, 25-500 nm+/−35 nm, 25-500 nm+/−40 nm, 25-500 nm+/−45 nm, or 25-500 nm+/−50 nm. In some embodiments, the nanoparticles (e.g. nanobeads) have a mean particle diameter of about 20 to about 200 nm.

In some embodiments, the magnetic particle may be a magnetic nanoparticle (e.g. nanobead) that is composed of oxides, such as ferrites, maghemite, magnetite, or iron oxide, optionally modified by surfactants, silica, silicones or phosphoric acid derivatives. In some embodiments, the nanoparticle (e.g. nanobead) is composed of ferrites with a shell (e.g. a silica shell, optionally modified). In some embodiments, the magnetic nanoparticle (e.g. nanobead) is metallic (e.g. iron, cobalt, etc.). In some embodiments, the magnetic nanoparticle (e.g. nanobead) is metallic with a shell (e.g. of gentle oxidation, surfactants, polymers and precious metals (e.g. of gold, graphene, etc.)).

In some embodiments, a particle described herein may be a nanoparticle (e.g. nanobead) that comprises one or more quantum dots. In some embodiments, the nanoparticle comprises a metal core and one or more quantum dots. In some embodiments, the nanoparticle comprises a metal core that may be studded with one or more quantum dots. In some embodiments, the nanoparticle comprises a metal core that may be studded with a plurality of quantum dots. Quantum dots are discrete nanoparticles that have properties similar to bulk semiconductors such that when exposed to electromagnetic energy they in turn emit energy. Quantum dots can be engineered to be sensitive to energy in the infrared region, the visible spectrum, and even ultraviolet range through changes in size and composition. Further, they can be designed to be either photoluminescent or photovoltaic, producing either light or energy, respectively.

In some embodiments, the reporter may be a nanoparticle (e.g. nanobead), which may comprise one or more quantum dots. In some embodiments, the reporter comprises a metal core and one or more quantum dots. In some embodiments, the reporter comprises a metal core that may be studded with one or more quantum dots. In some embodiments, the reporter comprises a metal core that may be studded with a plurality of quantum dots.

In some embodiments, the reporter may comprise one or more quantum dots. In some embodiments, the reporter conjugate may comprise one or more quantum dots. In some embodiments, the reporter and/or the reporter conjugate may comprise a plurality of quantum dots.

Examples of quantum dots, e.g. produced by colloidal methods, include, but are not limited to, cadmium-selenide (CdSe), cadmium-sulfide (CdS), indium-arsenide (InAs), and indium-phosphide (InP) cadmium-tellurium-sulfide (CdTeS). The number of atoms that comprise a quantum dot can range from 100 to 100,000, typically with a diameter ranging from 2 to 20 nm (e.g., 2, 3, 4, 5, 6, 7, 8, 9, 10, 11, 12, 13, 14, 15, 16, 17, 18, 19, 20, 2.5, 3.5, 4.5, 5.5, 6.5, 7.5, 8.5, 9.5, 10.5, 11.5, 12.5, 13.5, 14.5, 15.5, 16.5, 17.5, 18.5, 19.5, 20.5 nm).

In some embodiments, particle materials, including quantum dot materials, include, but are not limited to, carbon, colloidal gold, germanium, indium arsenide, indium antimonide, gallium arsenide, gallium nitride, cadmium/selenium/telluride, lead, lead oxide, lead sulfide, lead selenide, indium gallium phosphide, silicon, colloidal silver, mercury cadmium telluride, iron, iron oxide, cobalt, graphene, lanthanum, cerium, strontium carbonate, manganese, manganese oxide, nickel oxide, platinum, lithium, lithium titanate, tantalum, copper, palladium, molybdenum, boron carbide, silicon carbide, titanium carbide, tungsten oxide, aluminum, niobium, thulium, aluminum nitride, tin, aluminum oxide, tin oxide, antimony, dysprosium, paseodynium, antinmony oxide, erbium, rhenium, barium, ruthenium, beryllium, samarium, bismuth oxide, boron, gadolinium, boron nitride, vanadium oxide, strontium, ytterbium, zirconium, diamond (C), Silicon (Si), germanium (Ge), silicon carbide (SiC), silicon-germanium (SiGe), aluminium antimonide (AlSb), aluminium arsenide (AlAs), aluminium nitride (AlN), aluminium phosphide (AlP), boron nitride (BN), boron phosphide (BP), boron arsenide (BAs), gallium antimonide (GaSb), gallium arsenide (GaAs), gallium nitride (GaN), gallium phosphide (GaP), indium antimonide (InSb), indium arsenide (InAs), indium nitride (InN), indium phosphide (InP), aluminium gallium arsenide (AlGaAs), indium gallium arsenide (InGaAs, InxGai_ₓAs), indium gallium phosphide (InGaP), aluminum indium arsenide (AlInAs), aluminum indium antimonide (AlInSb), gallium arsenide nitride (GaAsN), gallium arsenide phosphide (GaAsP), aluminum gallium nitride (AlGaN), aluminum gallium phosphide (AlGaP), indium gallium nitride (InGaN), indium arsenide antimonide (InAsSb), indium gallium antimonide (InGaSb), aluminum gallium indium phosphide (AlGaInP, also InAlGaP, InGaAlP, AlInGaP), aluminum gallium arsenide phosphide (AlGaAsP), indium gallium arsenide phosphide (InGaAsP), aluminum indium arsenide phosphide (AlInAsP), aluminum gallium arsenide nitride (AlGaAsN), indium gallium arsenide nitride (InGaAsN), indium aluminium arsenide nitride (InAlAsN), gallium arsenide antimonide nitride (GaAsSbN), gallium indium nitride arsenide antimonide (GaInNAsSb), gallium indium arsenide antimonide phosphide (GaInAsSbP), cadmium selenide (CdSe), cadmium sulfide (CdS), cadmium telluride (CdTe), zinc oxide (ZnO), zinc selenide (ZnSe), zinc sulfide (ZnS), zinc telluride (ZnTe), cadmium zinc telluride (CdZnTe, “CZT”), mercury cadmium telluride (HgCdTe), mercury zinc telluride (HgZnTe), mercury zinc selenide (HgZnSe), cuprous chloride (CuCl), lead selenide (PbSe), lead sulfide (PbS), lead telluride (PbTe), tin sulfide (SnS), tin telluride (SnTe), lead tin telluride (PbSnTe), thallium tin telluride (Ti₂SnTe₅), thallium germanium telluride (Tl₂GeTe₅), bismuth telluride (Bi₂Te₃), cadmium phosphide (Cd₃P₂), cadmium arsenide (Cd₃As₂), cadmium antimonide (Cd₃Sb₂), zinc phosphide (Zn₃P₂), zinc arsenide (Zn₃As₂), zinc antimonide (Zn₃Sb₂), lead(II) iodide (Pbl₂), molybdenum disulfide (MoS₂), gallium selenide (GaSe), tin sulfide (SnS), bismuth sulfide (Bi₂S₃), copper indium gallium selenide (CIGS), platinum silicide (PtSi), bismuth(III) iodide (BiI₃), mercury(II) iodide (HgI₂), thallium(I) bromide (TlBr), titanium dioxide: anatase (TiO₂), copper(I) oxide (Cu₂O), copper(II) oxide (CuO), uranium dioxide (UO₂), uranium trioxide (UO₃), and the like.

In various embodiments, the magnetic field is applied using an external magnet. In various embodiments, the magnet is a permanent magnet (e.g. neodymium iron boron (NdFeB), samarium cobalt (SmCo), alnico, and ceramic or ferrite magnets). In various embodiments, the magnet is a temporary magnet. In various embodiments, the magnet is an electromagnet.

In various embodiments, the detection of the label is undertaken near the magnetic field. In various embodiments, the detection of the label is undertaken away from the magnetic field as in, for example, performed in a chamber that is separate from a chamber in which a magnetic pull down step is performed.

Immunoassay Kits that May be Used According to the Methods of the Invention

In some embodiments, the invention includes kits that include an immunoassay as described herein for practicing one or more methods described herein.

In some embodiments, the invention provides a pregnancy detection kit, which includes an immunoassay described herein, and involves the detection of hCG (e.g., β-hCG).

In some embodiments, the invention provides an ovulation detection kit, which includes an immunoassay described herein, and involves the detection of LH.

In some embodiments, the invention provides a pregnancy detection kit, which includes an immunoassay described herein, and involves the detection of E3G.

In some embodiments, the invention provides a kit for detecting PSA, which includes an immunoassay described herein.

In some embodiments, the invention provides a kit for detecting antibodies against HSV (e.g., HSV-1 and/or HSV-2), which includes an immunoassay described herein. In an embodiment, the kits described herein are specific for HSV-2 antibodies over HSV-1 antibodies.

In some embodiments, the invention provides a kit for detecting Strep-A, which includes an immunoassay described herein.

In other embodiments, the invention provides a kit for detecting infection by, for example, gonorrhea and chlamydia, which includes an immunoassay described herein.

In other embodiments, the invention provides a kit for detecting a disease or condition, such as, but not limited to, inflammation and diabetes, which includes an immunoassay described herein.

Unless defined otherwise, all technical and scientific terms used herein have the same meaning as is commonly understood by one of skill in the art to which this invention belongs. All patents and publications referred to herein are incorporated by reference in their entireties.

### Definitions

As used herein, “a,” “an,” or “the” means one or more than one.

Further, the term “about” when used in connection with a referenced numeric indication means the referenced numeric indication plus or minus up to 10% of that referenced numeric indication. For example, the language “about 50%” covers the range of 45% to 55%.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

As used herein, the term “sample” may refer to a solution, suspension, mixture, or undiluted amount of bodily fluid that may or may not include an analyte of interest. A sample, as used herein, may include water and/or a buffer. In some embodiments, the buffers described herein may be added to reduce or eliminate hook effects, which are present in most immunoassay platforms in the art. In some embodiments, a “large volume” of sample may be a 20 μL or greater volume of sample or 20 μL to 500 μL of sample. In some embodiments a “small volume” of sample may be less than 20 μL of sample or 1 μL to 15 μL of sample.

As used herein, the term “bodily fluid” may refer to any fluid that can be isolated from the body of an individual and includes, but is not limited to whole blood, plasma, serum, bile, saliva, urine, tears, perspiration, cerebrospinal fluid (CSF), semen, swabbed samples (e.g. cheek swabs, throat swabs, etc.), mucus, sputum, menstrual blood, menstrual fluid, vaginal mucus, amniotic fluid, synovial fluid, breast milk, ear wax, preejaculate, lochia, Rheum, lymph, pus, and the like. In some embodiments, bodily fluid may more particularly refer to whole blood, serum, urine, saliva, swabbed samples, mucus, or semen. In certain embodiments, bodily fluid may more particularly refer to whole blood, serum, urine, or saliva. In some embodiments, the bodily fluid may include an analyte of interest (e.g., a biomarker).

As used herein, the term “analyte of interest” or “target analyte” or “analyte” may be used be used interchangeably and refer to an antigen and/or a biomarker for a biological event, including any of the biomarkers described herein. In some embodiments, the biological events may include a disease event (e.g., disease biomarker), an inflammation event (e.g., an inflammation biomarker), a reproduction event (e.g., a reproduction biomarker), and/or an aging event (e.g., an aging biomarker). Disease biomarkers may include one or more disease biomarkers related to or associated with the onset of disease, the offset of disease, and/or the presence of a disease state in a patient. Disease biomarkers may include one or more of a viral biomarker, a bacterial biomarker, a cancer biomarker, or a symptom biomarker. Viral biomarkers may include, but are not limited to biomarkers for common cold (e.g. rhinovirus), influenza, herpes, Zika, and/or HIV. In some embodiments, viral biomarkers may include herpes simplex virus (HSV), one or more rhinovirus proteins, one or more influenza A/B/C proteins, one or more HSF-1/2 proteins, and/or one or more HIV virus proteins. Bacterial biomarkers may include, but are not limited to, biomarkers for strep throat (i.e., Streptococcus-A (Strep-A)), biomarkers for chlamydia, and/or biomarkers for gonorrhea. In some embodiments, bacterial biomarkers may include, but are not limited to, one or more streptococcus proteins, one or more Chlamydia trachomatis proteins, and/or one or more Neisseria gonorrhoeae proteins. Symptom biomarkers may include, but are not limited to, biomarkers for coughing, wheezing, runny nose, nausea, cramps, tightness of the chest, light-headedness, sore throat, and/or chest pain. Disease biomarkers may also include, but are not limited to, biomarkers for cardiac distress and/or diabetes. In some embodiments, disease biomarkers may include troponin, CRP, and/or ha1c. Cancer biomarkers may include biomarkers for prostate cancer, breast cancer, colorectal cancer, gastric cancer, GIST, leukemia/lymphoma, lung cancer, melanoma, and or pancreatic cancer. In some embodiments, prostate cancer biomarkers may include PSA. In some embodiments, breast cancer biomarkers may include one or more of ER/PR and HER-2/neu. In some embodiments, colorectal cancer biomarkers may include one or more of EGFR, KRAS, and UGT1A1. In some embodiments, gastric cancer biomarkers may include HER-2/neu. In some embodiments GIST biomarkers may include c-KIT. In some embodiments, leukemia/lymphoma biomarkers may include one or more of CD20 antigen, CD30, FIP1L1-PDGRFalpha, PDGFR, PML/RAR alpha, TPMT, and UGT1A1. In some embodiments, lung cancer biomarkers may include one or more of ALK, EGFR, and KRAS. In some embodiments melanoma biomarkers may include BRAF. Inflammatory biomarkers, which may include anti-inflammatory biomarkers, may include one or more inflammatory biomarkers described in U.S. Patent Application Publication No. 2010/0275282, the entirety of which is incorporated herein by reference. Reproduction biomarkers may include biomarkers for ovulation, fertilization, implantation, and/or embryo development. In some embodiments, reproduction biomarkers may include β-human Chorionic Gonadotropin (δ-hCG or hCG), hyperglycosylated hCG, luteinizing hormone (LH), estrone-3-glucuronide (E3G), early pregnancy factor (EPF), and/or pre implantation factor. Aging biomarkers or age-related biomarkers include one or more biomarkers described in U.S. Patent Application Publication No. 2008/0124752, the entirety of which is incorporated herein by reference. Additional antigens/biomarkers of interest include, but are not limited to, any known antigens/biomarkers associated with SARS, Hand foot and mouth disease, cardiac biomarkers, thyroid hormone, obesity biomarkers, biomarkers relating to bleeding disorders such as vWF, Factor 8, Factor 10, fifths disease, cold, flu, Ebola, E coli, listeria, and salmonella.

As used herein, the term “magnetic particle” refers to any particle having at least some magnetic characteristic, e.g., ferromagnetic, paramagnetic, and superparamagnetic property. The terms “bead” and “particle” may be used interchangeably. In some embodiments, a magnetic particle may include magnetic materials such as iron, nickel, and cobalt, as well as metal oxides such as Fe₃O₄, BaFe₁₂O₁₉, Mn₂O₃, Cr₂O₃, CoO, NiO, and CoMnP. In some embodiments, the magnetic particle contains, or fully consists of, a polymeric magnetic material. Polymeric magnetic material includes for example, material in which the magnetic material is mixed with polymeric material and magnetic material that is coated with polymeric material. Preferably the magnetic material is only one component of the microparticle whose remainder consists of a polymeric material to which the magnetically responsive material is affixed (see coded particles below). Exemplary methods for the preparation of or composition of magnetic particles are described in, e.g., U.S. Pat. Nos. 6,773,812 and 6,280,618, the entirety of which are incorporated herein by reference. In some embodiments, a magnetic particle may be a magnetic nanoparticle or magnetic microparticle, as described herein.

As used herein, the term “capture moiety” may refer to antibodies, single chain antibodies, antigen-binding antibody fragments, antigens, receptors, ligands, aptamers, aptamer receptors, nucleic acids, or small molecules that are conjugated or bound, or that may be conjugated or bound, to a magnetic particle that is selected to bind a target analyte. In certain embodiments, the capture moiety is a capture antibody.

As used herein, the term “capture antibody” may refer to an antibody conjugated or bound, or that may be conjugated or bound, to a magnetic particle that is selected to bind a target analyte.

As used herein, the term “reporter binding moiety” may refer to antibodies, single chain antibodies, antigen-binding antibody fragments, antigens, receptors, ligands, aptamers, aptamer receptors, nucleic acids, or small molecules that are conjugated or bound, or that may be conjugated or bound, to a reporter that is selected to bind a target analyte. In certain embodiments, the reporter binding moiety is a reporter antibody.

As used herein, the term “reporter antibody” may refer to an antibody conjugated or bound, or that may be conjugated or bound, to a reporter that is selected to bind a target analyte. In some embodiments, the “capture antibody” and “reporter antibody” may both bind different portions of the target analyte.

As used herein, the terms “reporter” and “label” may be used interchangeably, and may generally refer to a signal generating compound and/or detectable label or a core (e.g., a metal core) with one or more signal generating compounds and/or detectable labels connected to the core. In some embodiments, the reporter may be a fluorescent reporter, a phosphorescent reporter, or colorimetric reporter such as a colored particle for measuring absorbance and/or scattering of light (or, for example, the presence absence of a certain color through colorimetric analysis). In some embodiments, any suitable detectable label as is known in the art can be used. For example, the detectable label can be a radioactive label (such as ³H, ¹²⁵I, ³⁵S, ¹⁴C, ³²P, and ³³P), an enzymatic label (such as horseradish peroxidase, alkaline phosphatase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum or metal containing (Mc) dots (e.g., zinc sulfide-capped cadmium selenide), a thermometric label, or an immuno-polymerase chain reaction label. In various embodiments, the label includes without limitation fluorophores, chromophores, radioisotopes, magnetic particles, gold particles, enzyme substrates, and the like. In some embodiments, the label is a chemiluminescent or fluorescent protein, such as, for example, green fluorescent protein (GFP), enhanced green fluorescent protein (EGFP), Renilla reniformis green fluorescent protein, GFPmut2, GFPuv4, yellow fluorescent protein (YFP), enhanced yellow fluorescent protein (EYFP), cyan fluorescent protein (CFP), enhanced cyan fluorescent protein (ECFP), enhanced blue fluorescent protein (EBFP), citrine and red fluorescent protein from discosoma (dsRED), luciferase, umbelliferone, rhodamine, fluorescein, dichlorotriazinylamine fluorescein, dansyl chloride, phycoerythrin, and the like. In some embodiments, the label is a non-protein organic fluorophore of any of the following families: xanthene derivatives, such as fluorescein, rhodamine, Oregon green, eosin, and Texas red; cyanine derivatives, such as cyanine, indocarbocyanine, oxacarbocyanine, thiacarbocyanine, and merocyanine; squaraine derivatives and ring-substituted squaraines, including Seta, SeTau, and Square dyes; naphthalene derivatives (dansyl and prodan derivatives); coumarin derivatives; oxadiazole derivatives, such as pyridyloxazole, nitrobenzoxadiazole and benzoxadiazole; anthracene derivatives, such as anthraquinones, including DRAQ5, DRAQ7 and CyTRAK Orange; pyrene derivatives, such as cascade blue, etc.; oxazine derivatives, such as Nile red, Nile blue, cresyl violet, oxazine 170, etc.; acridine derivatives, such as proflavin, acridine orange, acridine yellow, etc.; arylmethine derivatives, such as auramine, crystal violet, malachite green; and tetrapyrrole derivatives, such as porphin, phthalocyanine, bilirubin. In various embodiments, the label includes without limitation enzymatic labels, e.g., enzymes such as horseradish peroxidase, alkaline phosphatase, beta-galactosidase, glucose 6-phosphate dehydrogenase, and the like. In some embodiments, the reporter may be a quantum dot as described herein. In some embodiments, the reporter may comprise a quantum dot as described herein. In some embodiments, the reporter may include a metal core (i.e., gold core) with a silica shell, wherein the silica shell is impregnated with a plurality (e.g., 100-600) quantum dots.

Although the open-ended term “comprising,” as a synonym of terms such as including, containing, or having, is used herein to describe and claim the invention, the invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

While preferred embodiments of the invention are shown and described herein, such embodiments are provided by way of example only and are not intended to otherwise limit the scope of the invention. Various alternatives to the described embodiments of the invention may be employed in practicing the invention.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Reporter/Antibody Coupling Procedure

A covalent coupling procedure was developed to couple antibodies to fluorescently labeled reporter particles. The reporter particles described, unless otherwise noted, are metal core quantum dot studded particles (Nanocompsix)), which have a carboxyl surface coating.

### Experimental Procedure

Sonicate the glass vial containing the stock reporter particles for 10 seconds by holding the vial still in the sonicator. Mix the reporter particles using a P1000 set to 600 until no pellet or settling is noticeable.

Transfer 600 μL of the Stock reporters to a 1.5 mL VWR Centrifuge Tube, using a P1000 set to 600.

Add 10 μL each, using a P10 set to 10, to three 1 mg tubes of 1-ethyl-3-(3-dimethylaminopropyl)carbodiimide (EDC). Place the EDC tubes into the Ezee Mini-Centrifuge. Spin for 5 seconds. Remove the 10 uL from two of the tubes and add to the third. Mix well with pipette.

Add 28.8 μL of the EDC Solution to the 600 μL of reporter particles using a P100 set to 28.8.

Add 20 μL each, using a P20 set to 20, to three 2 mg tubes of N-hydroxysulfosuccinimide (Sulfo-NHS). Mix well by pipette until no solids are visible. Remove the 20 μL from two of the tubes and add to the third. Mix well with pipette.

Add 57.6 μL of the EDC Solution to the 600 μL of reporter using a P100 set to 57.6.

With a P200 Pipette set to 200 μL mix the reporter particle-EDC-NHS mixture.

Place the reporter particle-EDC-NHS mixture in the bath Sonicator and turn on for 1 minute.

Place the reporter particle-EDC-NHS on the Rugged Rotator set to 40% speed and rotate end-over-end for 15 minutes.

In a 1.5 mL Eppendorf Protein Lo-bind tube, add 360 μL 1 mg/mL Antibody. If the antibody is above 1 mg/mL dilute to 1 mg/mL using MB Coupling Buffer (i.e., PBS, 0.01% Tween-20, pH 7.4).

Spin the reporter particle-EDC-NHS solutions in a centrifuge at 3600 rcf for 5 minutes.

With a P200, carefully remove the supernatant from the tube. Make sure not to disturb the pellet of reporter particles at the bottom of the tube.

With a P1000 add 600 μL of MD Reaction Buffer to the reporter particle pellet.

Place the reporter particle tube in the Sonicator for 10 seconds.

Using a P1000, set to 600 μL, mix well and transfer the 600 μL of reporter particles to the tube containing the antibody.

Place the reporter particle-Antibody solution onto the rugged rotator set to 40% speed and rotate for 1 hour.

Make a 1:10 Dilute Hydroxylamine solution by adding 7 μL of Stock Hydroxylamine Solution using a P10 set to 7 to 63 μL NF-Water as measured by P100.

With a P100 add 60 μL of 1:10 Dilute Hydroxylamine solution to the reporter particle-Antibody solution.

Place the reporter particle-Antibody solution onto the rugged rotator set to 40% speed and rotate for 10 minutes.

Spin the reporter particle-Antibody solutions in a centrifuge at 3600 rcf for 5 minutes.

With a P200 carefully remove the supernatant from the tube. Make sure not to disturb the pellet of reporter particles at the bottom of the tube.

With a P1000 add 600 μL of MB Storage Buffer to the reporter particle pellet. Mix thoroughly and sonicate for 10 seconds.

Spin the reporter particle-Antibody solutions in a centrifuge at 3600 rcf for 5 minutes.

With a P200, carefully remove the supernatant from the tube. Make sure not to disturb the pellet of reporter particles at the bottom of the tube.

With a P1000, add 600 μL of MB Storage Buffer to the reporter particle pellet. Mix thoroughly and sonicate 10 seconds. Spin the reporter particle-Antibody solutions in a centrifuge at 3600 rcf for 5 minutes.

With a P200 carefully remove the supernatant from the tube. Make sure not to disturb the pellet of reporter particles at the bottom of the tube.

With a P1000 add 300 μL of MB Storage Buffer to the reporter particle pellet. Mix thoroughly and sonicate 10 seconds.

Add a volume of MB Storage Buffer (i.e., PBS, 0.01% Tween-20, 0.05% Sodium Azide, pH 7.4) to the coupled reporter particles via appropriately sized pipette, and ensure that the coupled reporter particles are at 2.2 moles particles/L.

Store antibody coupled reporter particles at 4° C. for up to one month.

### Magnetic Bead/Antibody Coupling Procedure

This procedure describes an exemplary protocol for covalently coupling antibodies to magnetic capture particles (i.e., magnetic beads).

The magnetic beads used in this protocol are nanoparticles with a superparamagnetic Fe₂O₃ core and a biocompatible outer coating. The surface is activated with carboxyl groups.

### Experimental Procedure

### Protein Preparation

Using a 7K Zeba Desalting Column, buffer exchange the antibody of interest into MB Coupling Buffer (i.e., PBS, 0.01% Tween-20, pH 7.4); if the concentration of antibody is greater than 1 mg/mL dilute the antibody solution to 1 mg/mL using MB coupling buffer before using the Zeba Column. Measure IgG Concentration.

### Magbead Cleaning and Resuspension

Using a P200 set to 175, add 175 μL Ocean Nanotech Super Carboxyl Magbeads twice to a 0.5 mL protein Lo-Bind Eppendorf tube resulting in 350 μL.

Place tube onto a Promega Magstand for 1 minute.

Remove the supernatant by using a P200 set to 200 twice (supernatant may be brown-colored).

Using a P200 set to 100, add 100 μL Magbead Activation Buffer and resuspend completely by pipetting gently (no dark regions should be visible).

Place Tube back onto Promega Magstand for 1 minute.

Remove the supernatant by using a P200 Set to 200 twice (Supernatant may be brown-colored).

Using a P200 set to 100, add 100 μL Magbead Activation Buffer (i.e., MES, 0.01% Tween-20, pH 6.0) and resuspend completely by pipetting gently (no dark regions should be visible).

Place tube onto tube float and sonicate for 1 minute.

### Magbead Activation

Using a P100 set to 100, add 100 μL nuclease-free water to a Pierce no-weigh 1 mg EDC tube (10 mg/mL).

Vortex for 30 seconds, and spin on tube spinner for 30 seconds.

Using a P100 set to 25, add 25 μL of the suspended EDC to the magnetic bead solution.

Add 200 μL nuclease-free water into the ThermoFisher no-weigh 2 mg Sulfo-NHS tube and mix well to dissolve the solids (10 mg/mL).

Using a P100 set to 25, add 25 μL of the suspended Sulfo-NHS to the magnetic bead solution.

Place tube onto rotator and react at room temperature for 15 minutes.

### Antibody Conjugation

Using a P200 set to 150, transfer the 150 μL activated Magbeads into a new Eppendorf lo-bind tube.

Place the tube onto a Promega Magstand for 30 seconds.

Remove the supernatant using a P200 set to 200.

Remove tube from mag stand and using a P200 add an amount of activation buffer. This amount changes based on antibody concentration to ensure same final incubation antibody concentration batch-to-batch.

Hold tube halfway submerged in the sonicator for 10 seconds to resuspend.

Using a P100, add an amount of buffer-exchanged strep antibody and mix tube well using a P200 set to 200. This amount changes based on antibody concentration to ensure same final incubation antibody concentration batch-to-batch.

React at room temperature for 2.5 hours on the rotator. Mix with a P200 pipette at the 1:15 time point.

Using a P100 set to 50, add 50 μL Magbead Quenching Buffer.

React at room temperature for 30 minutes on the rotator.

Place the tube onto a Promega Magstand for 1 minute.

Remove the supernatant using a P200 set to 200.

Remove tube from Magstand and using a P200 set to 200, add 200 μL Magbead Storage Buffer and pipette gently to resuspend.

Place the tube onto a Promega Magstand for 1 minute.

Remove the supernatant using a P200 set to 200.

Remove tube from Magstand and using a P200 set to 200, add 200 μL Magbead Storage Buffer and pipette gently to resuspend.

Place the tube onto a Promega Magstand for 1 minute.

Remove the supernatant using a P200 set to 200.

Remove tube from Magstand and using a P200 set to 150, add 300 μL Magbead Storage Buffer by pipetting twice, pipette gently to resuspend

Hold tube halfway submerged in the sonicator for 10 seconds to fully resuspend.

Store coupled Magbeads at 4° C. for up to one month.

### Magnetic Bead/Biomarker Coupling Procedure (Estrone-3-Glucuronide-Magnetic Beads)

This procedure describes an exemplary protocol for covalently coupling a biomarker (i.e., estrone-3-glucoronide (E3G) to magnetic capture particles (i.e., magnetic beads).

The magnetic beads used were nanoparticles with a superparamagnetic Fe₂O₃ core and a biocompatible outer coating. The surface is activated with amine groups.

### Magnetic Bead Cleaning and Resuspension

Using a P1000 set to 300 μL, pipette Ocean Nanotech Super Amine Magnetic Beads to a 0.5 mL protein Lo-Bind Eppendorf tube.

Place tube onto a Promega Magstand for 1 minute.

Remove the supernatant by using a P200 Set to 200 twice (supernatant may be brown-colored).

Using a P100 set to 90, add 90 μL Magbead Activation Buffer (i.e., MES, 0.01% Tween-20, pH 6.0) and resuspend completely by pipetting gently (no dark regions should be visible).

Place Tube back onto Promega Magstand for 1 minute.

Remove the supernatant by using a P100 Set to 100 once (supernatant may be brown-colored).

Using a P100 set to 90, add 90 μL Magbead Activation Buffer and resuspend completely by pipetting gently (no dark regions should be visible).

Place tube onto tube float and sonicate for 1 minute.

### Reagent Mixing

Using a P100 set to 100, add 100 μL nuclease-free water to a Pierce no-weigh 1 mg EDC tube. (10 mg/mL)

Vortex for 30 seconds, and spin on tube spinner for 30 seconds.

Using a P100 set to 30, add 30 μL of the suspended EDC to the magnetic bead solution.

Add 200 μL nuclease-free water into the ThermoFisher no-weigh 2 mg Sulfo-NHS tube and mix well to dissolve the solids (10 mg/mL).

Using a P100 set to 30, add 30 μL of the suspended Sulfo-NHS to the magnetic bead solution.

Using a P100 set to 30 add 30 μL of 1 mg/mL E3G solution in NF-Water.

### Activation Incubation

Place tube onto rotator and react at room temperature for 15 hours.

### Magnetic Bead Cleaning and Final Suspension

Place the tube onto a Promega Magstand for 1 minute.

Remove the supernatant using a P200 set to 200 twice.

Remove tube from Magstand and using a P200 set to 200, add 200 μL Magbead Storage Buffer (i.e., PBS, 0.01% Tween-20, 0.05% sodium azide, pH 7.4) and pipette gently to resuspend.

Place the tube onto a Promega Magstand for 1 minute.

Remove the supernatant using a P200 set to 200.

Remove tube from Magstand and using a P200 set to 200, add 200 μL Magbead Storage Buffer and pipette gently to resuspend.

Place the tube onto a Promega Magstand for 1 minute.

Remove the supernatant using a P200 set to 200.

Remove tube from Magstand and using a P1000 set to 300, add 300 μL Magbead Storage Buffer, pipette gently to resuspend.

Hold tube halfway submerged in the sonicator for 10 seconds to fully resuspend.

Store at 4° C. for up to one month.

### Example 1—Immunoassay Sandwich Mode Operation

An immunoassay as described herein is prepared for use in sandwich mode, which is an ideal setup for processing small fluid volumes.

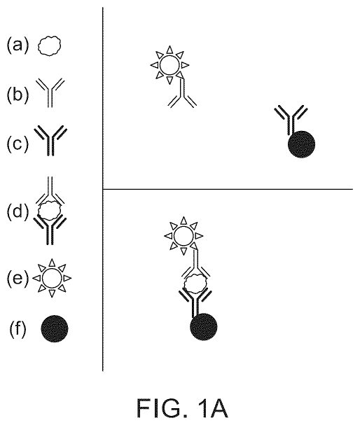

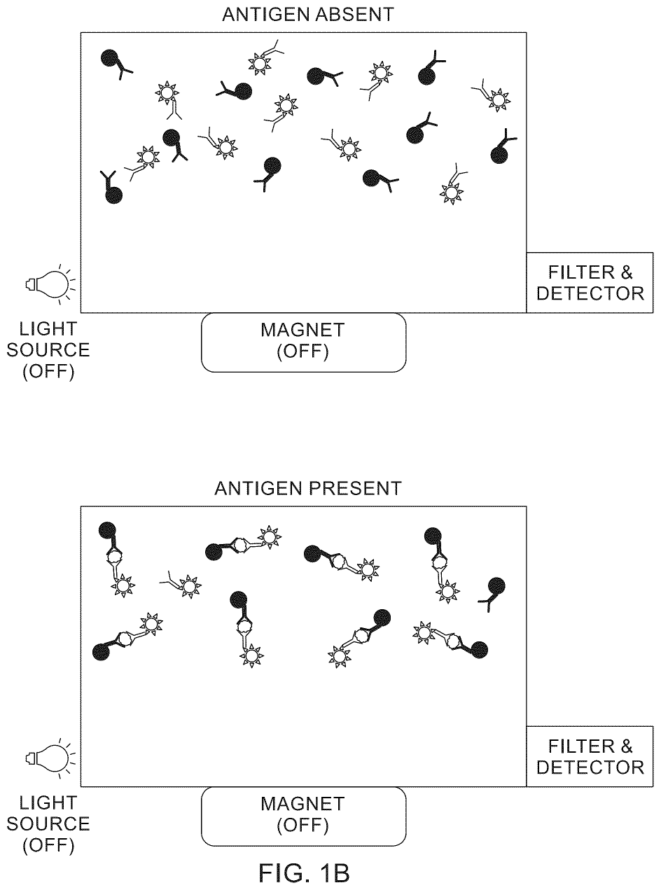

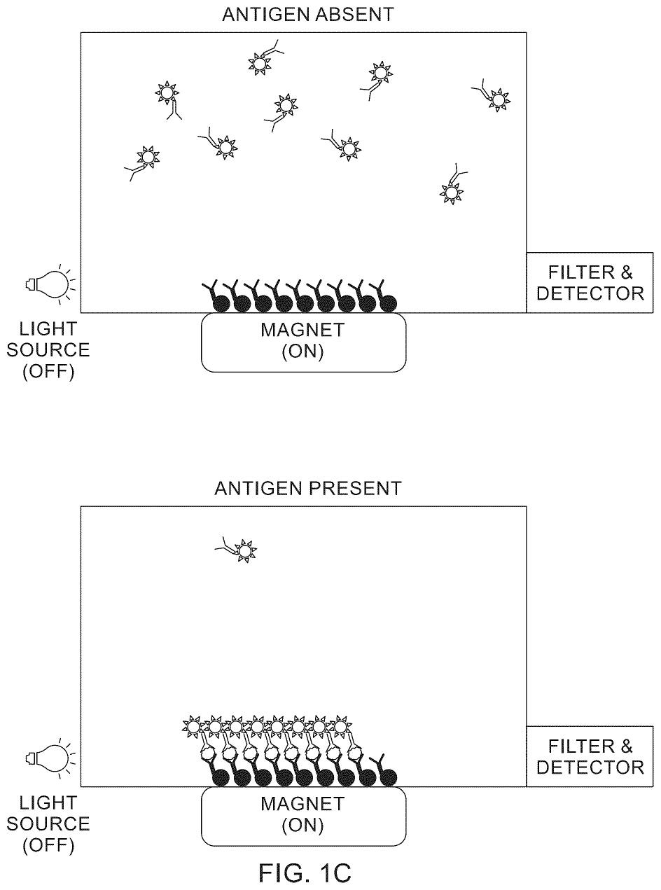

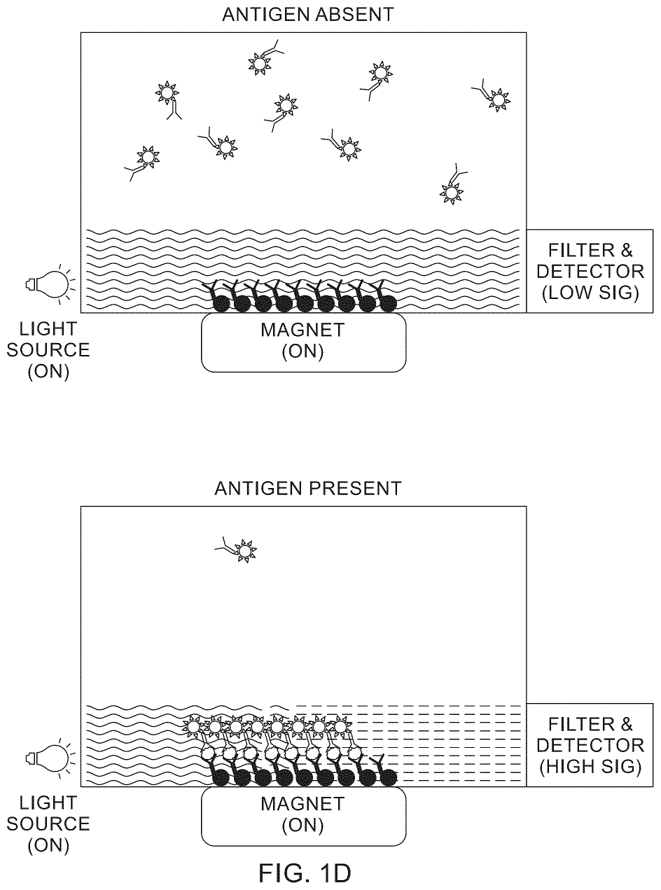

As shown in FIGS. 1A to 1D, a sandwich mode for an immunoassay described herein may be used. As shown therein, a reporter conjugate including a reporter (gold core particles with a silica shell impregnated with 100-600 quantum dots (nanoComposix)) and an antibody #1 and a magnetic conjugate including an antibody #2 and a magnetic particle may be used detect an hCG analyte of interest in an analysis chamber (FIG. 1A).

In a first step, the magnetic conjugate and the reporter conjugate may be added to the assay chamber and mixed with the sample containing the analyte of interest (hCG) (FIG. 1B). A magnetic field may be applied (a “pulldown”) by a magnet to separate the analyte of interest from the sample (FIG. 1C). Light may then be transmitted through a portion of the analysis chamber to cause the reporter to fluoresce (FIG. 1D). Such fluorescence may be detected by the detector.

In the absence of analyte, the reporter will not be pulled down with the analyte and no fluorescence will occur.

### Example 2—Immunoassay Separate Addition Operation

An immunoassay as described herein is prepared for use in separate addition mode, which is a preferred setup for processing large volumes of samples and allowing for concentration of the analyte. This leads to greatly improved sensitivity.

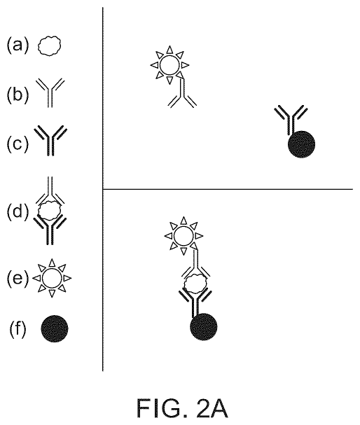

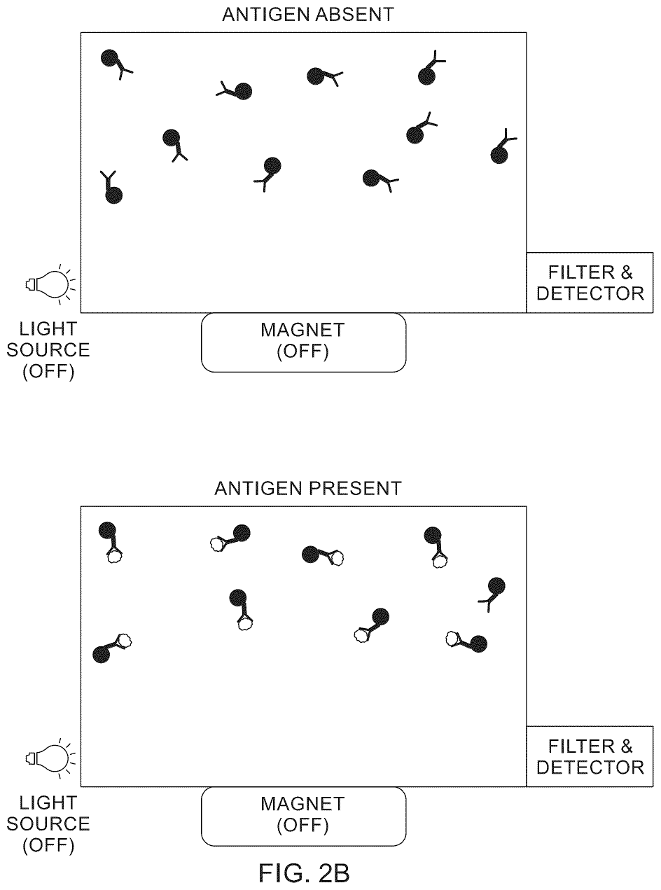

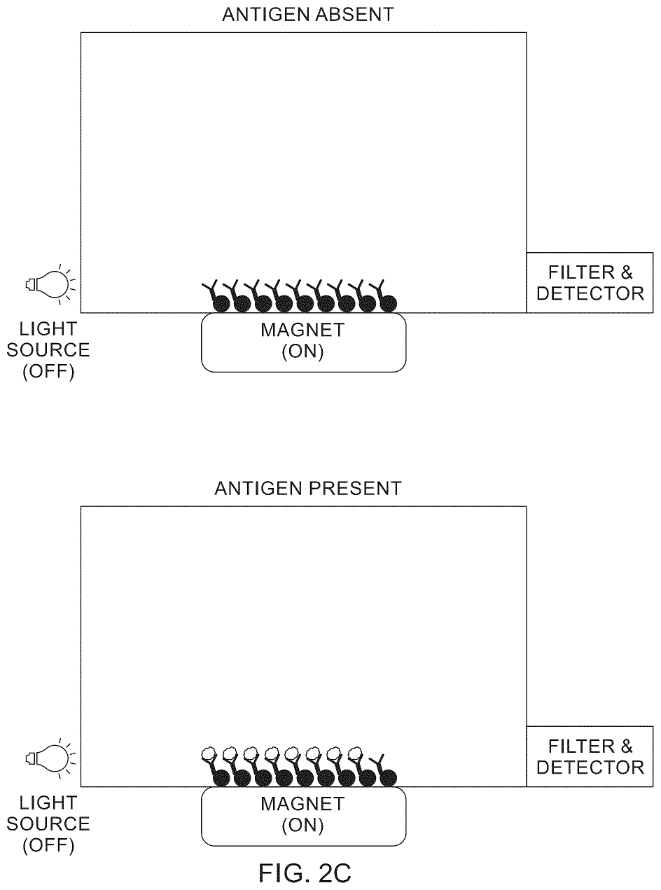

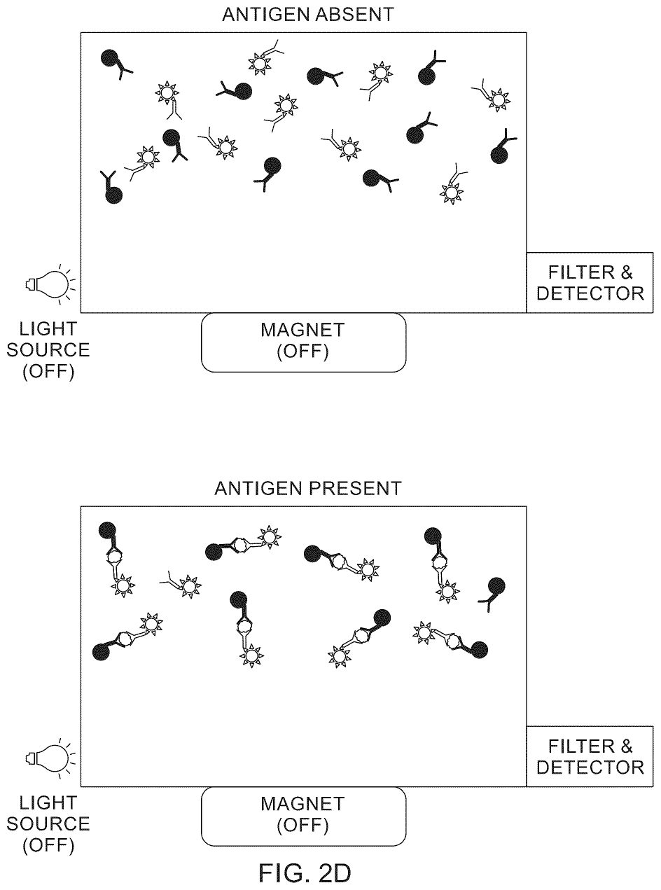

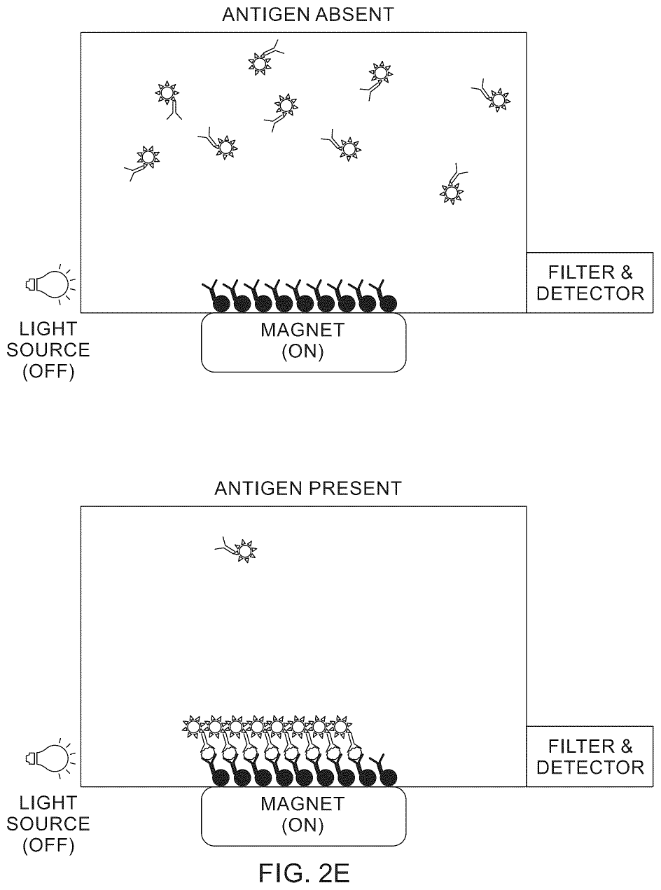

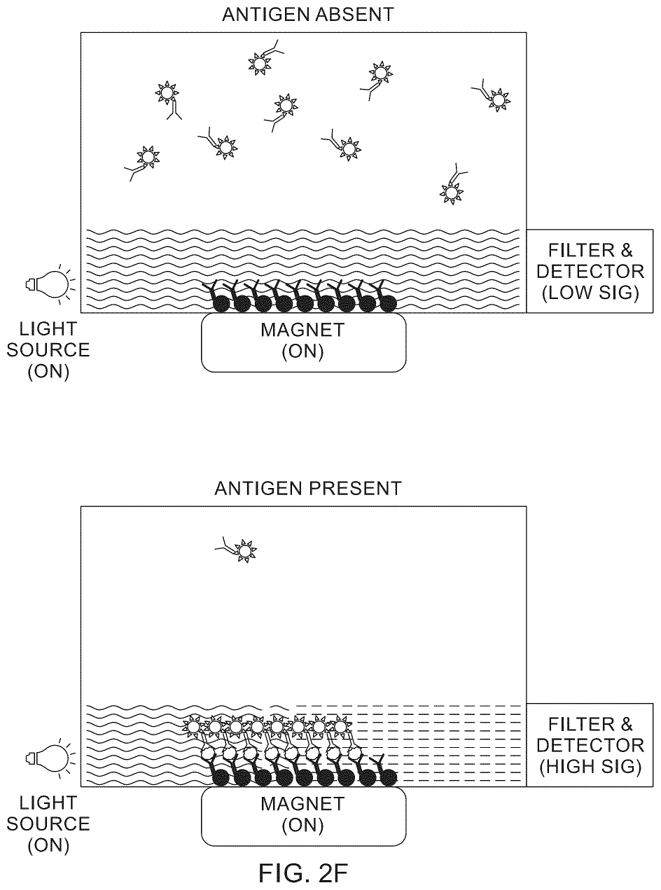

As shown in FIGS. 2A to 2F, a separate addition mode for an immunoassay described herein may be used. As shown therein, a reporter conjugate including a reporter (gold core particles with a silica shell impregnated with 100-600 quantum dots (nanoComposix)) and an antibody #1 and a magnetic conjugate including an antibody #2 and a magnetic particle may be used to detect an hCG analyte of interest in an analysis chamber (FIG. 2A).

In a first step, the magnetic conjugate may be added to the assay chamber and mixed with the sample containing the analyte of interest (hCG) (FIG. 2B). A pulldown may be performed and, optionally, a volume of sample may be removed and an additional volume of sample may then be added (FIG. 2C). After additional sample is added, the magnetic field may be deactivated and the magnetic conjugate may again be mixed with the sample. This process may be repeated to concentrate the analyte of interest. After concentrating the analyte of interest, the reporter conjugate may be added and mixed with the sample to bind to the analyte of interest (FIG. 2D). An additional pulldown may be performed to separate the analyte of interest from the sample (FIG. 2E). Light may then be transmitted through a portion of the analysis chamber to cause the reporter to fluoresce (FIG. 2F). Such fluorescence may be detected by the detector.

In the absence of analyte, the reporter will not be pulled down with the analyte and no fluorescence will occur.

### Example 3—Competitive Immunoassay Operation—First Method

An immunoassay as described herein is prepared for use in competitive mode, which is a preferred setup where the analyte of interest is too small for binding by two antibodies. This may also be preferred where only one antibody exists for binding the analyte of interest.

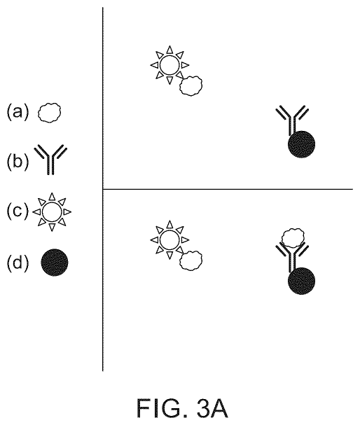

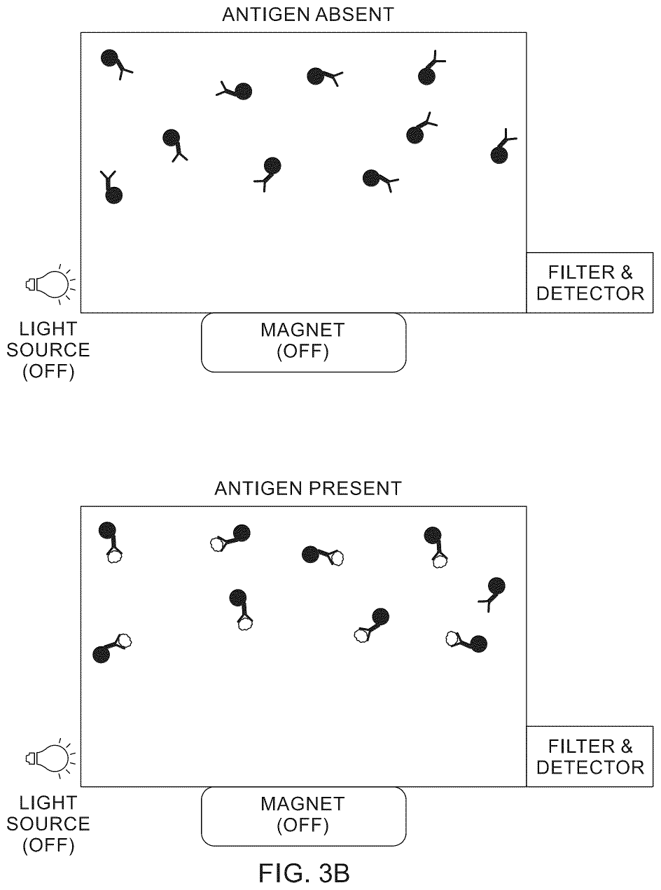

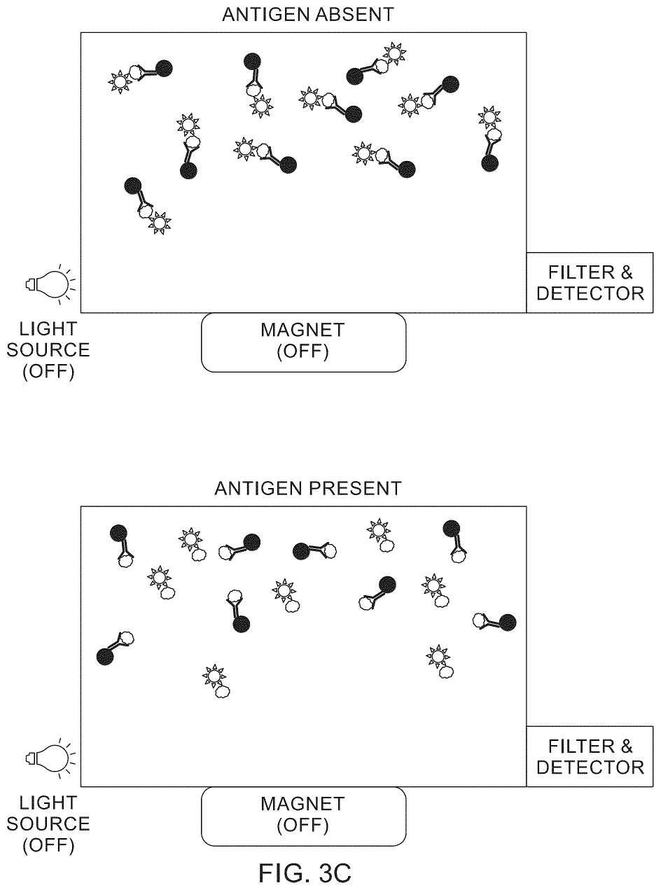

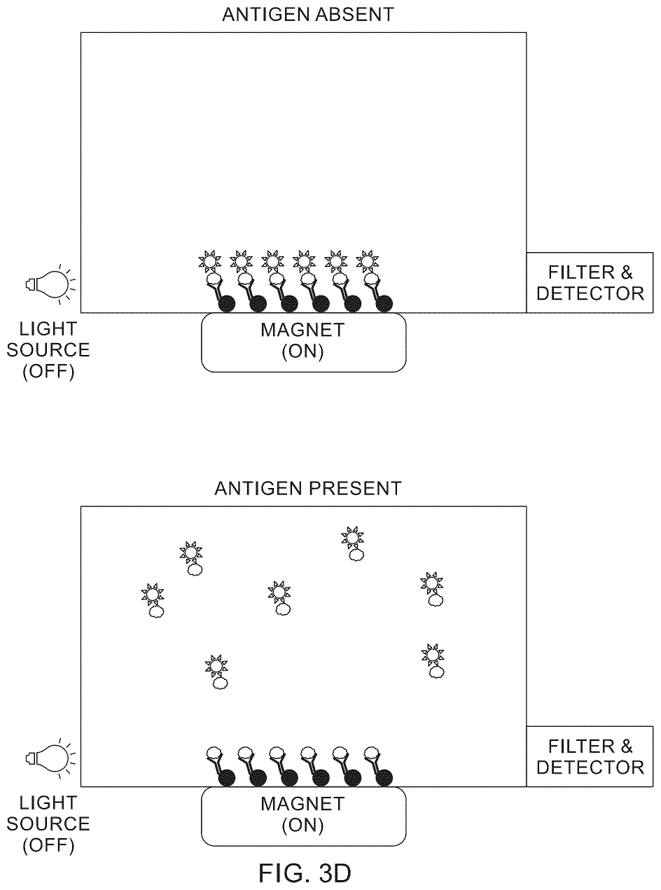

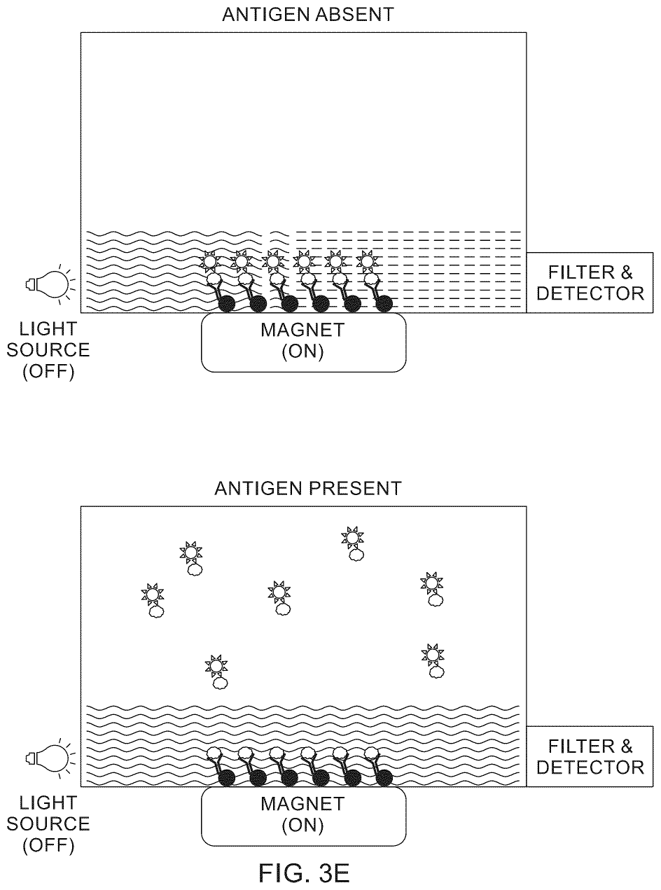

As shown in FIGS. 3A to 3E, a competitive mode for an immunoassay described herein may be used. As shown therein, a reporter-labeled analyte and a magnetic conjugate including an antibody and a magnetic particle may be used to detect an analyte of interest in an analysis chamber (FIG. 3A).

In a first step, the magnetic conjugate may be added to the assay chamber and mixed with the sample containing the analyte of interest (FIG. 3B). The reporter-labeled analyte may then be added and mixed with the sample (FIG. 3C). When the analyte of interest is present, the reporter-labeled analyte does not bind to the magnetic conjugate. Indeed, the binding site on the magnetic conjugate's antibody is occupied with the analyte of interest from the sample. However, if the analyte of interest is not present in the sample, the magnetic conjugate will bind to the reporter-labeled analyte (FIG. 3C). Performing a pulldown to separate the analyte of interest from the sample (FIG. 3D). Light may then be transmitted through a portion of the analysis chamber to cause the reporter to fluoresce (FIG. 3E). Such fluorescence may be detected by the detector.

In the absence of analyte, the reporter-labeled analyte will be pulled down with magnetic conjugate and fluorescence will be detected.

### Example 4—Competitive Immunoassay Operation—Second Method

An immunoassay as described herein is prepared for use in an alternative competitive mode

A magnetic particle may be used with an analyte of interest bound thereto. In that respect, the magnetic particle bears the analyte of interest at the start of the assay rather than the reporter as in Example 3. The reporter conjugate includes an antibody and a reporter that may be used to detect the analyte of interest in the sample in an analysis chamber.

In a first step, the reporter conjugate is added to the analysis chamber and mixed with the sample containing the analyte of interest. The magnetic particles with analyte bound thereto are added to the analysis chamber and mixed with the sample. A pulldown is then performed to separate the magnetic particles with analyte bound thereto from the sample. The sample is then removed. Light may then be transmitted through the sample to cause the reporter to fluoresce. Such fluorescence may be detected by the detector.

In the absence of analyte, the reporter conjugate will be pulled down and will not be present in the sample, resulting in no fluorescence.

### Example 5—Tertiary Immunoassay Operation

An immunoassay as described herein is prepared for use in tertiary mode, makes use of three binding events to enhance the kinetics of the system. The tertiary binding motif can be applied to the sandwich mode (Example 1), the separate addition (Example 2), and the competitive assay modes (Example 3 and 4).

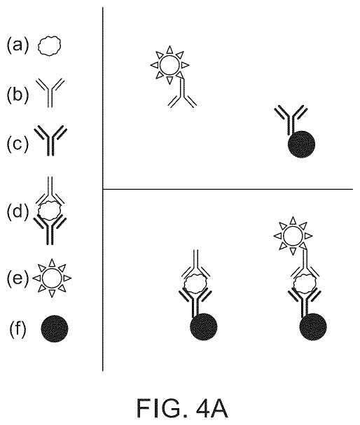

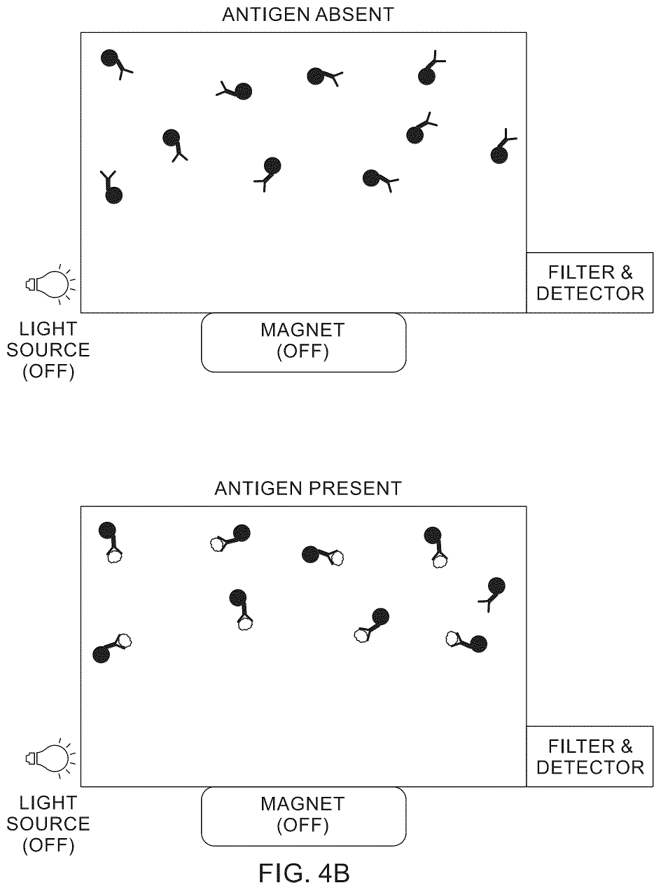

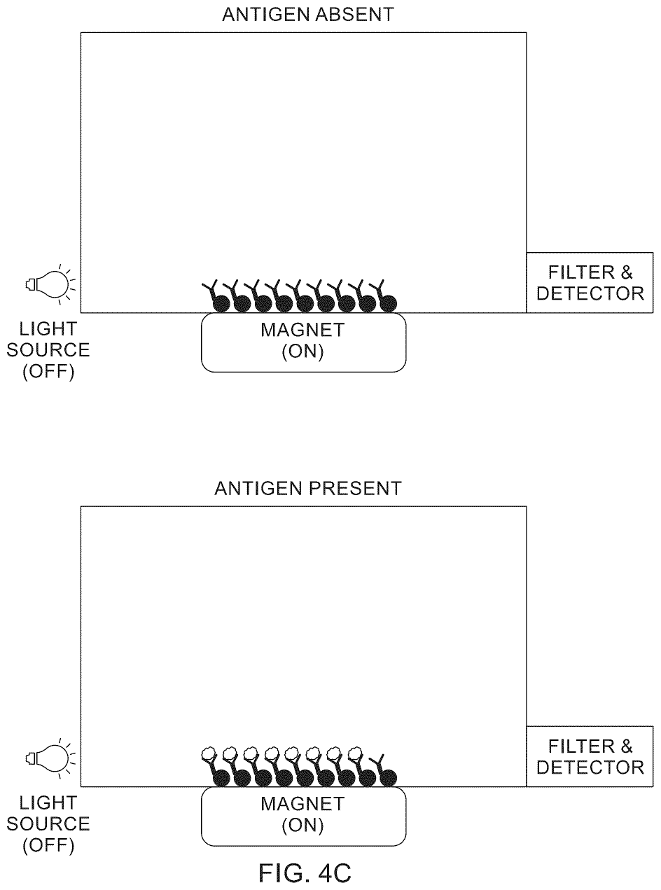

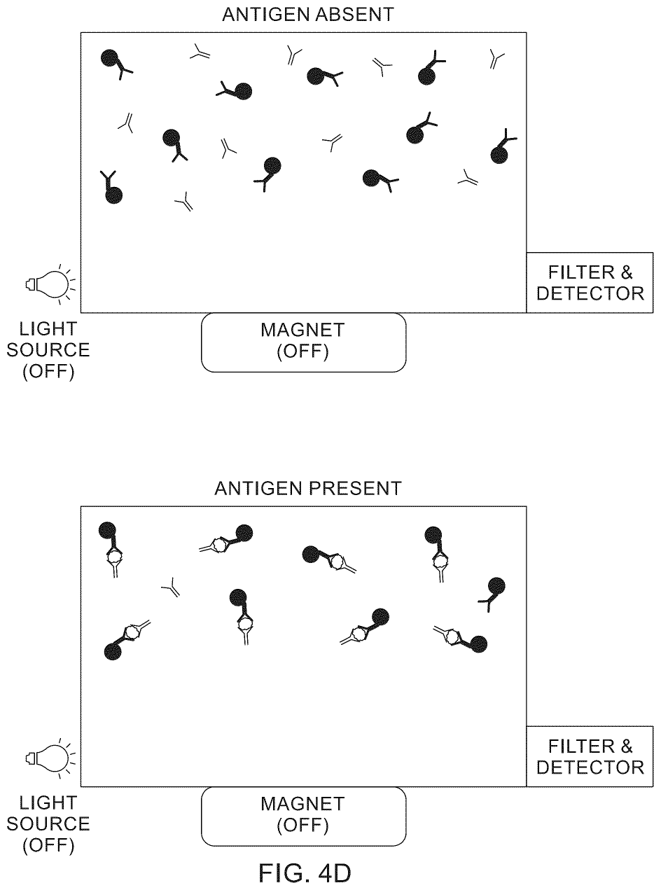

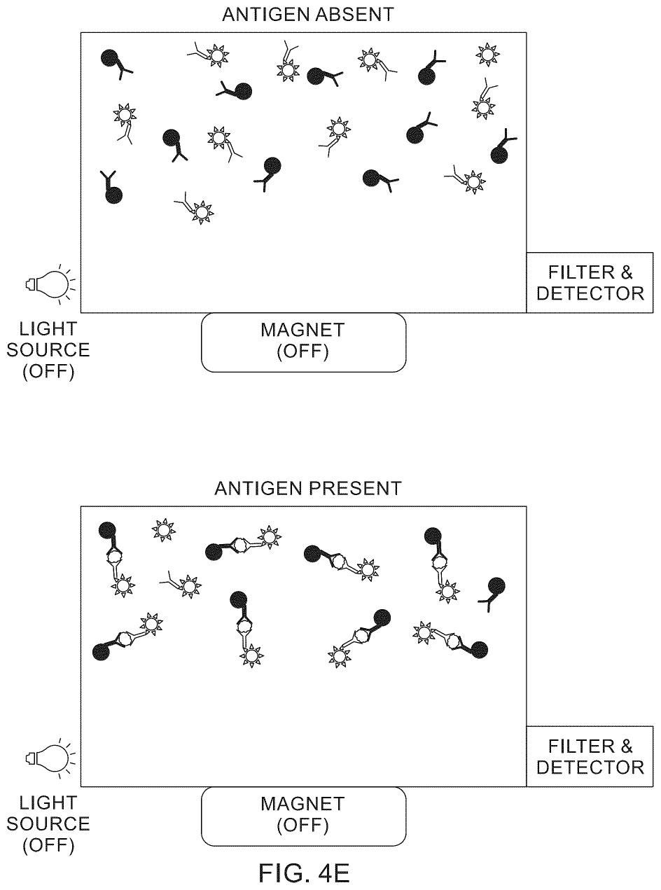

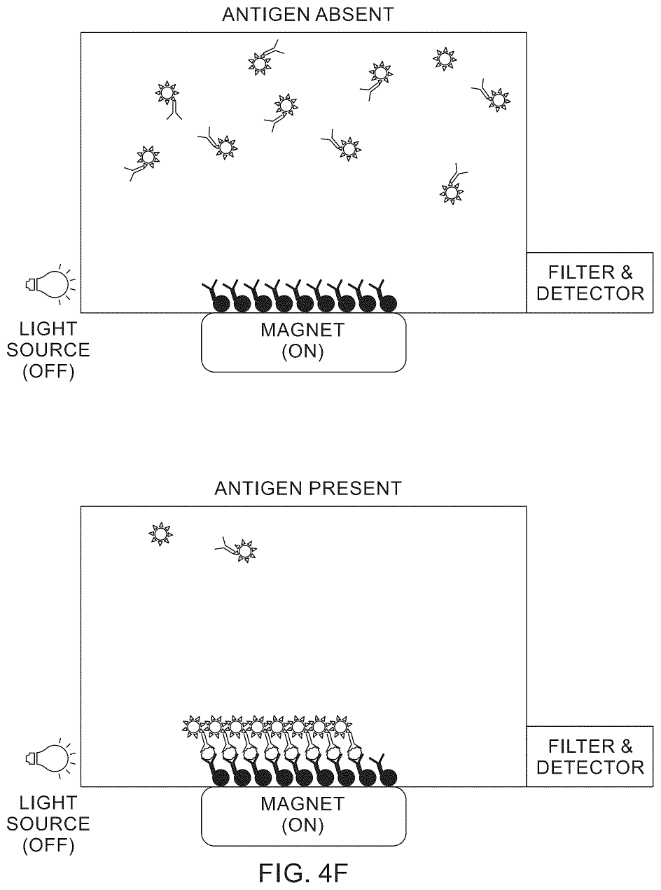

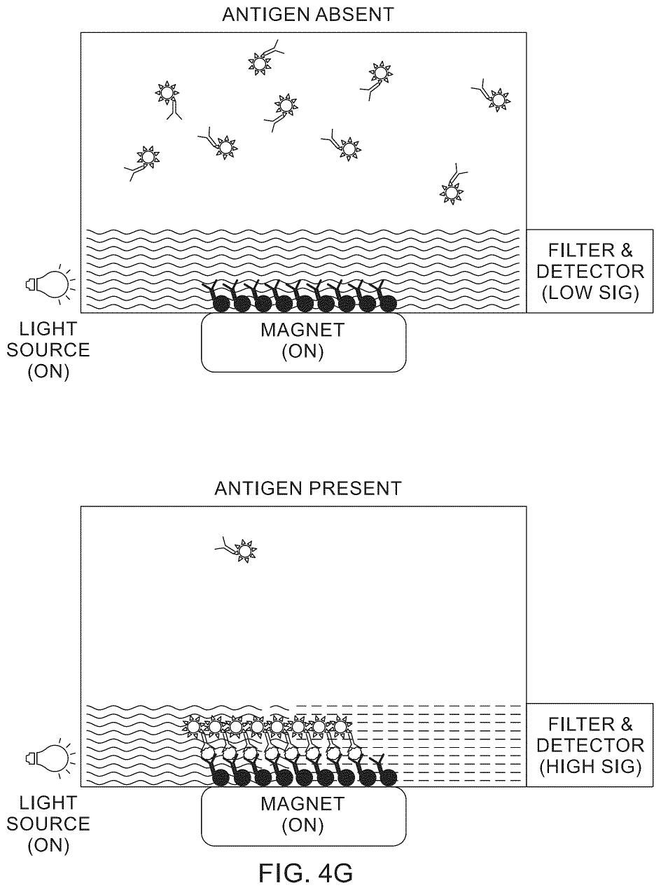

As shown in FIGS. 4A to 4G, a tertiary mode for an immunoassay described herein may be used. As shown therein, a reporter conjugate including a reporter (fluorescent quantum dot functionalized with streptavidin) and an antibody #1 (labeled with a biotin) and a magnetic conjugate including an antibody #2 and a magnetic particle may be used detect an hCG analyte of interest in an analysis chamber (FIG. 4A).

In a first step, the magnetic conjugate may be added to the assay chamber and mixed with the sample containing the analyte of interest (FIG. 4B). A magnetic field may be applied (a “pulldown”) by a magnet to separate the analyte of interest from the sample (FIG. 4C). Volumes of sample may be removed and analyte concentration steps may be performed as described in Example 2. The magnetic field may be deactivated and antibody #1 may be added to the analysis chamber, which may then bind to the analyte of interest that is also bound to the magnetic conjugate (FIG. 4D). The reporter may then be added to the analysis chamber, which may then bind to antibody #1 through the streptavidin-biotin binding interaction (FIG. 4E). A pulldown may then be performed to again separate the analyte from the sample (FIG. 4F).

Light may then be transmitted through a portion of the analysis chamber to cause the reporter to fluoresce (FIG. 4G). Such fluorescence may be detected by the detector.

In the absence of analyte, the reporter will not be pulled down with the analyte and no fluorescence will occur.

### Example 6—Demonstration of an Assay for Detecting Human Chorionic Gonadotropin

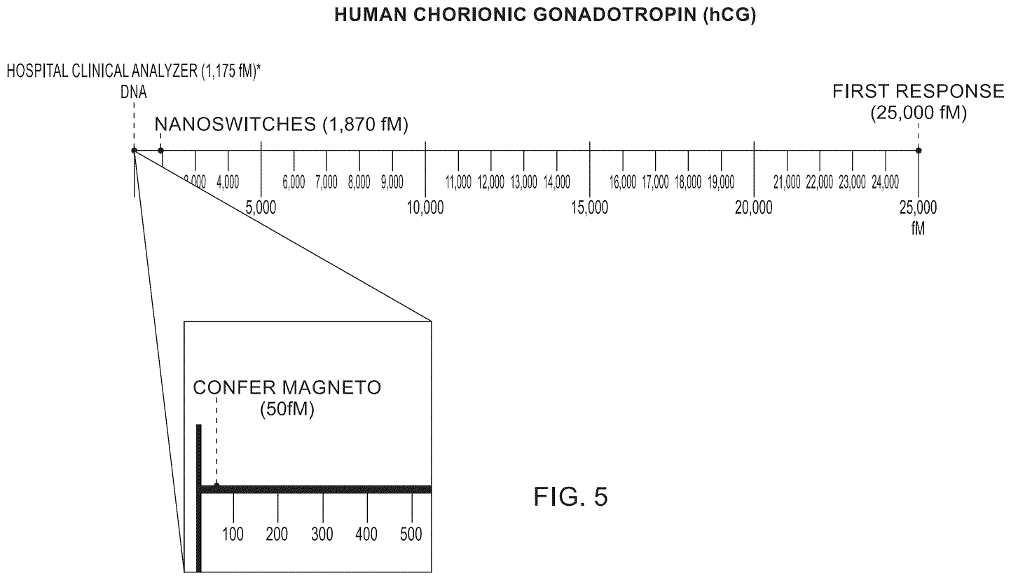

An immunoassay was provided for detecting human Chorionic Gonadotropin (hCG) in a urine sample using the protocol of Example 15. The assay exhibited femtomolar scale sensitivity as shown in FIG. 5.

### Example 7—Demonstration of an Assay for Detecting Luteinizing Hormone

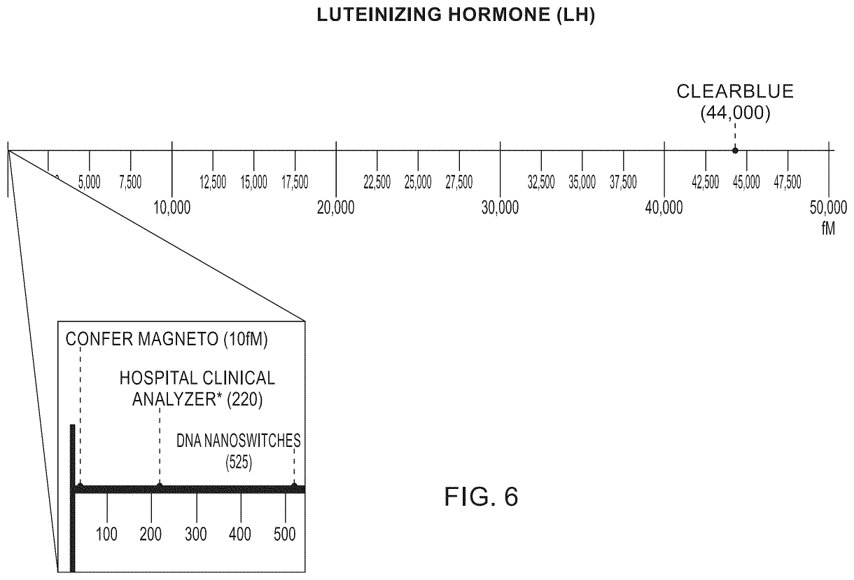

An immunoassay was provided to detect Leuteinizing Hormone (LH) in a urine sample. The assay exhibited femtomolar scale sensitivity as shown in FIG. 6.

The protocol used for detecting LH in urine is as follows below.

The magnetic beads in this assay are magnetic nanoparticles coated with an antibody that binds LH (Medix 5304).

The reporter in this assay is a fluorescently labeled nanoparticle, which has been coated with an antibody that binds LH (Medix 5304).

### Equipment Used

The following equipment was used.

Equipment
CPX2800 Ultrasonic Digital Bench Top Cleaner
Rugged Rotator: #099A-RD4612
SpectraMax i3x
Pipetman P200 Pipette
Pipetman P100 Pipette
Pipetman P20 Pipette
Pipetman P10 Pipette
Promega Magnetic Separation stand (0.5 mL)
Promega Magnetic Seperation stand (0.5 mL)
Timer

### Experimental Procedure

Before the day of collection (e.g., about 5 pm before the day of collection), dilute the magnetic beads and reporters in Chon Block, according to the following formulas, recording exact volumes used:

$$Volume of Reporters ( uL ) = Ceiling ( ( \frac{( Number of Subjects ) \times ( 3 Replicates ) \times ( 3 uL per Replicate ) + ( 36 uL for Calibration )}{( 51 X Dilution Factor )} ) \times ( 1.2 X Safety Factor ) )$$

$$Volume of ChonBlock ( uL ) = ( Volume of magnetic beads ) \times ( 51 X Dilution Factor ) - ( Volume of magnetic beads )$$

$$Volume of magnetic beads ( uL ) = Ceiling ( ( \frac{( Number of Subjects ) \times ( 3 Replicates ) \times ( 3 uL per Replicate ) + ( 36 uL for Calibration )}{( 6 X Dilution Factor )} ) \times ( 1.2 X Safety Factor ) )$$

$$Volume of ChonBlock ( uL ) = ( Volume of magnetic beads ) \times ( 6 X Dilution Factor ) - ( Volume of magnetic beads )$$

Sonicate reporters by holding the tube in the sonicator for 10 s. Mix reporters using a P100 set to 100.

Add the calculated volume of Chon Block to an 0.5 mL Eppendorf Protein Lo-bind tube using a an appropriately sized pipette.

Add the calculated volume of reporters to the Chon Block. Pipette up and down to ensure the pipette has been cleared of reporters.

Mix the now-diluted reporters with an appropriately sized pipette.

Sonicate the magnetic beads by holding the tube in the sonicator for 10 s. Mix magnetic beads using a P100 set to 100.

Add the calculated volume of Chon Block to an 0.5 mL Eppendorf Protein Lo-bind tube using an appropriately sized pipette.

Add the calculated volume of magnetic beads to the ChonBlock. Pipette up and down to ensure the pipette has been cleared of magnetic beads.

Mix the now-diluted magnetic beads with an appropriately sized pipette.

Store both diluted reagents at in a metal rack at 4° C. Reagents are good for 20 hours.

While the urine sample is being collected, allocate 3 μL magnetic beads to three 0.5 mL Eppendorf Protein Lo-bind tubes pre-labeled with the subject number, cycle date and replicate number (1,2,3), using a P10 pipette set to 3 μL.

When the sample arrives, add 3 μL of Urine to the First replicate tube.

Immediately after, start the timer, counting up in minutes.

After 30 seconds, with a P10 pipette add 3 μL of reporter to the First replicate tube. Mix with a P10 set to 3 μL.

As the timer hits 1 minute, using a P10 pipette add 3 μL of urine into second replicate tube. Mix with a P10 set to 3 μL.

As 1:30 seconds elapses on the timer, with a P10 pipette add 3 μL of reporters to the second replicate tube. Mix with a P10 set to 3 μL.

As the timer hits 2 minutes, using a P10 pipette add 3 μL of urine into third replicate tube. Mix with a P10 set to 3 μL.

As 2:30 seconds elapses on the timer, with a P10 pipette add 3 μL of reporters to the third replicate tube. Mix with a P10 set to 3 μL.

After 5 minutes elapse on the timer, place the first replicate tube on the Promega magnet stand and check as timer counts up by 1 minute.

At the 6-minute time point, place the second replicate tube on the second Promega magnet stand.

At 6:05, with a P20 Pipette set to 20 μL remove the supernatant from the First replicate tube, while keeping the tube on the magnet stand.

While keeping the tube on the magnet stand, add with a P200 pipette 100 μL of TBS to the First replicate tube, and then remove the 100 μL of TBS by over-pipetting.

While keeping the tube on the magnet stand, add with a P200 pipette another 100 μL of TBS to the First replicate tube, and then remove the 100 μL of TBS by over-pipetting.

Remove the Eppendorf tube from the magnet stand.

With a P20 pipette add 20 μL of TBS, and mix thoroughly by pipetting up and down.

At the 7-minute time point, place the third replicate tube on the First Promega magnet stand.

At 7:05, with a P20 Pipette set to 20 μL remove the supernatant from the second replicate tube, while keeping the tube on the magnet stand.

While keeping the tube on the magnet stand, add with a P200 pipette 100 μL of TBS to the second replicate tube, and then remove the 100 μL of TBS by over-pipetting.

While keeping the tube on the magnet stand, add with a P200 pipette another 100 μL of TBS to the second replicate tube, and then remove the 100 μL of TBS by over-pipetting.

Remove the Eppendorf tube from the magnet stand.

With a P20 pipette add 20 μL of TBS, and mix thoroughly by pipetting up and down.

At 8:05, with a P20 Pipette set to 20 μL remove the supernatant from the second replicate tube, while keeping the tube on the magnet stand.

While keeping the tube on the magnet stand, add with a P200 pipette 100 μL of TBS to the second replicate tube, and then remove the 100 μL of TBS by over-pipetting.

While keeping the tube on the magnet stand, add with a P200 pipette another 100 μL of TBS to the second replicate tube, and then remove the 100 μL of TBS by over-pipetting.

Remove the Eppendorf tube from the magnet stand.

With a P20 pipette add 20 μL of TBS, and mix thoroughly by pipetting up and down.

Store the assay tubes in a metal rack in the cooler for later analysis.

### Spectra Max Analysis

With a P20 Pipette Load 20 μL of each replicate sample onto the 384-well plate.

With a P10 Pipette Load 20 μL of TBS into 2 wells on the same 384-well plate.

Load the 384-well plate into the SpectraMax.

### Example 8—Demonstration of an Assay for Detecting Prostate Specific Antigen

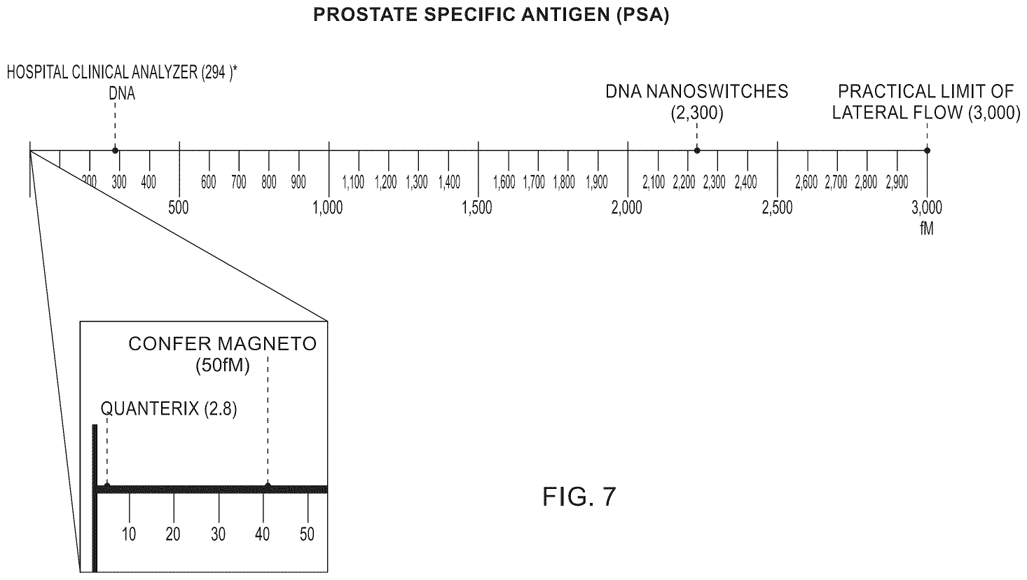

An immunoassay was provided to detect prostate specific antigen (PSA) in a serum/whole blood sample. The assay exhibited femtomolar scale sensitivity as shown in FIG. 7.

An exemplary protocol used for detecting PSA in whole blood and serum is as follows below.

The magnetic beads in this assay are magnetic nanoparticles coated with an antibody that binds PSA (Medix Anti-h PSA 8311). A solution of magnetic beads was prepared mixing and sonicating the magnetic beads for 10 s. A 200 pM magnetic bead solution includes 2.5 μL of magnetic beads and 22.5 μL of Chon Block.

The reporter in this assay is a fluorescently labeled nanoparticle, which has been coated with an antibody that binds PSA (Medix Anti-h PSA 8301)

### PSA Stripping Antibody Solution

PSA 8301 ab: Stock 5.3 mg/mL (35.35 μM). For 6.66 μM, 5.65 μL of Stock 8301 Antibody and 24.35 μL Chon Block were combined. For 660 nM, add 10 μL of 6.66 μM of the solution and 90 μL of Chon Block.

For the serum stripping solution, add 2 μL of the 660 nM solution and 98 μL of Chon Block.

For the whole blood stripping solution, add 10 μL of the 660 nM solution and 90 μL of Chon Block.

### Serum Preparation

Standard serum preparation included adding 20 μL of serum to 80 μL of Chon Block.

PSA stripped serum was prepared by adding 20 μL of serum to 80 μL of serum stripping solution.

### Whole Blood Preparation

Standard whole blood preparation included combining 76 μL of whole blood, 19 μL of Chon Block, and 4.75 μL of 10% Triton X-100.

PSA stripped whole blood was prepared by combining 76 μL of whole blood, 19 μL of whole blood stripping solution, and 4.75 μL of 10% Triton X-100.

### Incubation

Incubation was performed by first adding 25 μL of magnetic beads and 9 μL of reporters to 100 μL of prepared serum, and then incubating the sample for 30 minutes.

Incubation was also performed by first adding 25 μL of magnetic beads and 9 μL of reporters to 100 μL of whole blood, and then incubating the sample for 30 minutes.

### Pull Down After Incubation

A pull down was performed by exposing the incubated solutions to a magnet for 5 minutes.

The result of the pulldown was rinsed twice with 100 μL of PBS, while still on the magnet and resuspended with 27 μL of PBS.

The resuspended magnetic beads/reporters could then be analyzed by fluorescence spectroscopy to detect PSA in the whole blood and serum. The difference in signal between the standard serum (or whole blood) and the stripped serum (or whole blood) will give a measure of PSA levels that removes the matrix effects of the individual's serum.

### Example 9—Bacteria Immunoassay Operation

An immunoassay as described herein is prepared for use in detecting bacteria in a sample.

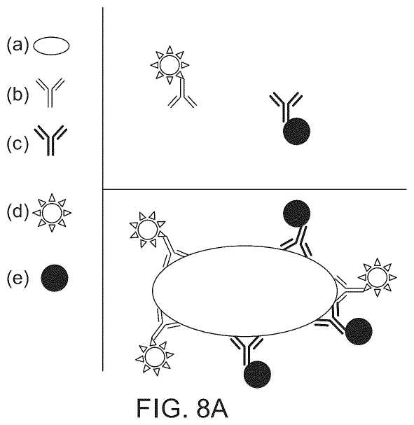

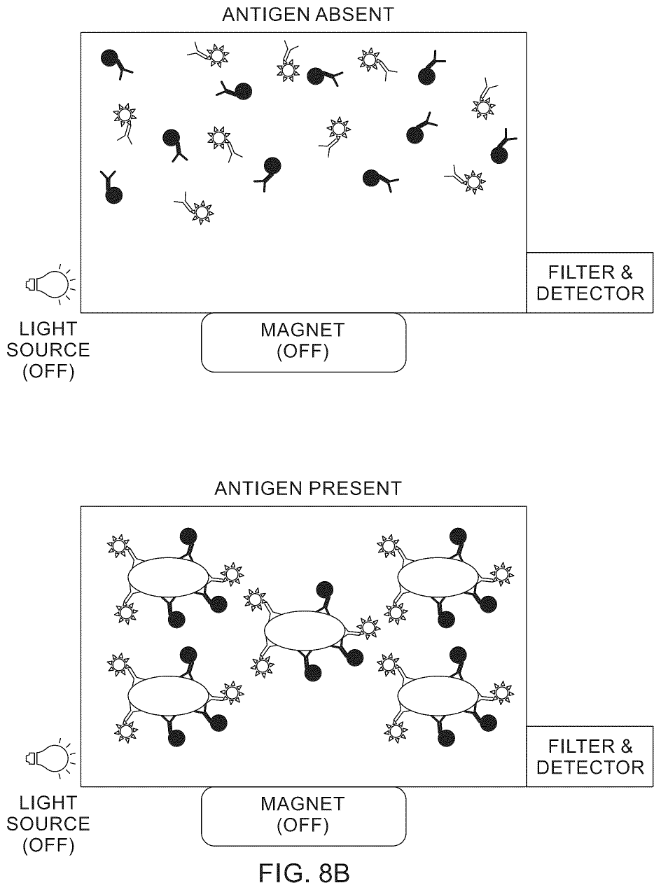

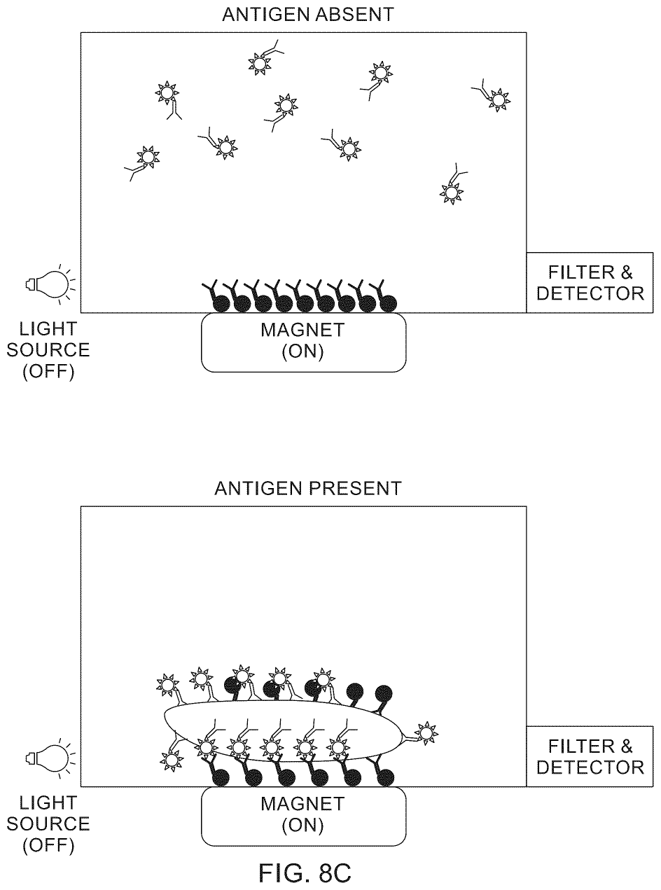

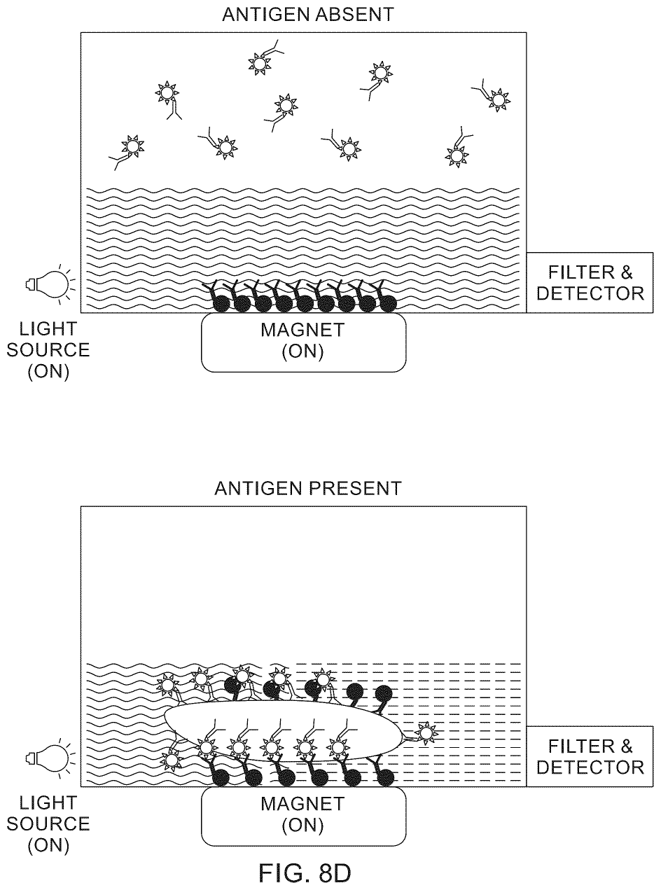

As shown in FIGS. 8A to 8C, a sandwich mode for an immunoassay described herein may be used to detect bacteria (e.g., Strep A) in a sample. As shown therein, a reporter conjugate including a reporter (gold core particles with a silica shell impregnated with 100-600 quantum dots (nanoComposix)) and an antibody #1 and a magnetic conjugate including an antibody #2 and a magnetic particle may be used detect a bacteria analyte of interest in an analysis chamber (FIG. 8A).

In a first step, the magnetic conjugate and the reporter conjugate may be added to the assay chamber and mixed with the sample containing the analyte of interest (bacteria) (FIG. 8B). A magnetic field may be applied (a “pulldown”) by a magnet to separate the analyte of interest from the sample (FIG. 8C). Light may then be transmitted through a portion of the analysis chamber to cause the reporter to fluoresce (FIG. 8D). Such fluorescence may be detected by the detector.

In the absence of analyte, the reporter will not be pulled down with the analyte and no fluorescence will occur.

### Example 10—Demonstration of a Bacteria Immunoassay in Detecting Group Strep A Bacteria

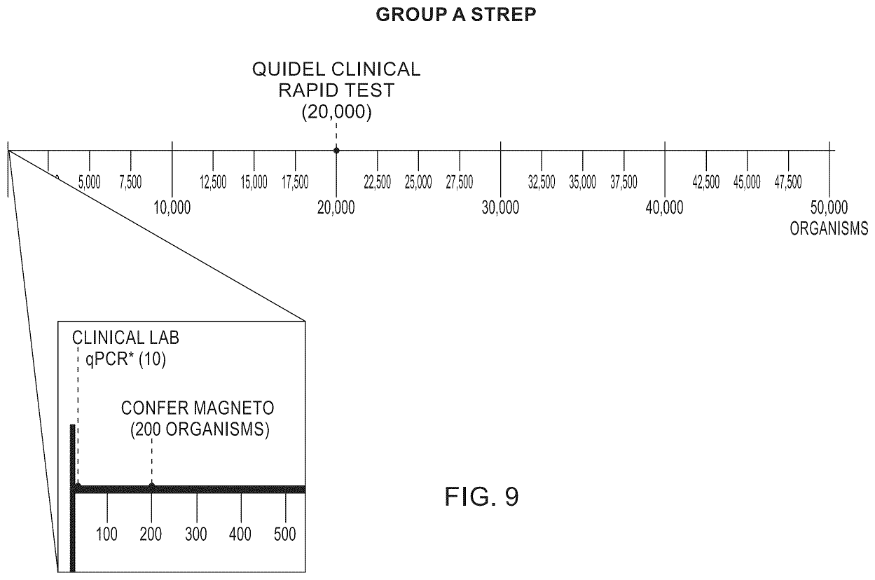

A bacteria immunoassay was provided as in Example 9, which was used to detect the presence of Strep A in a sample including throat, cheek, and saliva samples from 26 subjects with Strep throat and 15 subjects suspected to not be infected with Group A strep. The assay exhibited 200 organism scale sensitivity as shown in FIG. 9.

Furthermore, with regard to sensitivity, the present assay showed a sensitivity of 93% ND a specificity of 100% as compared to the Quidel Clinical grade test, which is 92% sensitive and 98% specific.

An assay used for detecting strep is as follows.

The magnetic beads in this assay are magnetic nanoparticles coated with an antibody that binds to Strep A (Biospacific G47091041). A 300 μM solution of magnetic beads was prepared by adding 4 μL of magnetic beads (2.89 nM) to a flask, pulling them down with a magnet, then resuspending in 38.5 μL of Chon Block

The reporter used in this assay is a fluorescently labeled nanoparticle, which has been coated with an antibody that binds to Strep A (Biospacific G47091041). A 250 μM solution of reporters was prepared by combining 4.5 μL of reporters (2.2 nM) and 35.5 μL of Chon Block.

### Stock Blocking Mixture Preparation

Biospacific G47091041 (1 m/mL) was prepared as a stock solution at 6.67 μM and 1 μM, 500 nM, and 100 nM solutions were prepared in PBS.

### Swab Processing

In a first step, a subject's tonsils are swabbed. 150 μL of PBS is then added to a squeezable tube. The swab is then submerged in the PBS solution. After 1 minute, the swab is pulled midway up the tube and pinched with fingers to extract the liquid from the swab.

A strep solution is prepared by combining 16 μL of swab processed mix and 2 μL of Chon Block.

A blocked strep solution is prepared by combining 16 μL of swab processed mix and 2 μL of 500 nM Strep blocking mixture.

### Sample Incubation

Strep Incubation: 16 μL of Strep Solution is combined with 3 μL of magnetic beads and 3 μL of reporters. Incubation proceeds for 10 minutes.

Blocked Incubation: 16 μL of Blocked Strep Solution is combined with 3 μL of magnetic beads and 3 μL of reporters. Incubation proceeds for 10 minutes.

### Pull Down After Incubation

The incubated solutions are then pulled down on a magnet for 5 minutes. The resulting pulldowns are rinsed twice with 100 μL of PBS, while still on the magnet, and then suspended with 27 μL of PBS.

The resuspended magnetic beads/reporters could then be analyzed by fluorescence spectroscopy to detect Strep A in a sample. The difference in signal between signal of the strep incubation tube and the blocked incubation tube will give a measure of Group A Streptococcus that removes the matrix effects from swabs of different individuals.

### Example 11—Method for a Competitive E3G Detection Assay

E3G can be detected in a sample analyzed by a competitive immunoassay described herein.

E3G Coupled magnetic beads and Anti-E3G Coupled Quantum Dots (gold core particles with a silica shell impregnated with 100-600 quantum dots (nanoComposix)) can be used. Buffers may be used in the analysis chamber, which may include chonblock and PBS. The Quantum Dot working solution may include 1 μL of Stock Anti-E3G Quantum Dots and 6 μL of Chonblock.

The protocol may be as follows:
a. Mix 6.25 μL of urine with 1.25 μL Quantum Dot Working Solution;
b. Incubate 5 minutes;
c. Add 5 μL of E3G Coupled Magnetic Beads;
d. Incubate 30 minutes;
e. Place on Magnet stand for 1 minute;
f. Remove Supernatant;
g. Rinse twice with 50 μL of PBS;
h. Resuspend in 27 μL PBS; and
i. Pipette 20 μL into well plate.

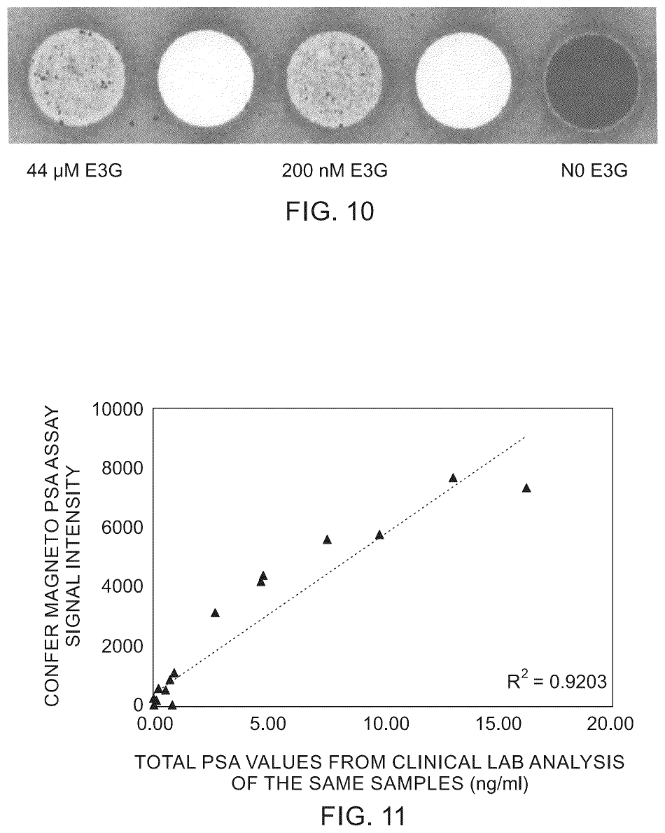

The pipetted material may then be imaged as shown in FIG. 10 where 44 μM and 200 nM E3G were detected in two samples as compared to blanks.

### Example 12—Clinical Study Measuring Correlation Between Measurements of Described Immunoassays and Measurements Performed in a Clinical Laboratory

A clinical study was performed measuring the correlation between measurements on the present PSA detecting immunoassay platform described in Example 8 and measurements done at a clinical laboratory showing very high correlation as seen in FIG. 11.

### Example 13—Clinical Validation of β-hCG Detection with Immunoassay Technology

In this example, the methods and standard operating procedures used in the clinical validation of the 3-hCG detection platform are described.

An IRB approved clinical study was launched that received nearly 500 inquiries, resulted in 76 enrolled subjects, 15 of whom became pregnant. 13 out of the 15 pregnant subjects had urine collected using the following frozen sample protocol, and 2 out of the 15 had samples collected using the fresh protocol.

Frozen Sample Protocol. Subjects collecting frozen samples were provided with urine cups, labels, Ziploc bags, and a sample log so that they could collect daily samples. The subjects would collect urine in the urine cup each morning, log the time of urination, label the cup, bag it, and immediately put it in their freezer. Upon completion of their cycle (either by getting a positive pregnancy test result using one of the provided tests, or having their period) the samples would be picked up from the subject's home by study staff who would retrieve the samples and transport them to the lab in a cooler filled with ice-packs. Samples were immediately racked and stored in our laboratory freezers (for details see materials and methods).

Fresh Sample Protocol. Subjects collecting fresh samples were provided with a single urine cup and a small thermos. The subjects would collect urine each morning and place the urine cup in the thermos. They would then send a text message to the clinical research coordinator who would bring the sample in for analysis within 2 hours of urine collection.

Sample Testing Paradigm. Samples were tested to find the first day on which First Response yielded a positive result (i.e. First Response reads negative on the day before, but positive on that day's urine). This day was termed the First Response Day. The immunoassay platform was then tested on the First Response Day, and at least three days prior. Additionally, two baseline days (days before the participant ovulates ensuring that she cannot be pregnant) were tested.

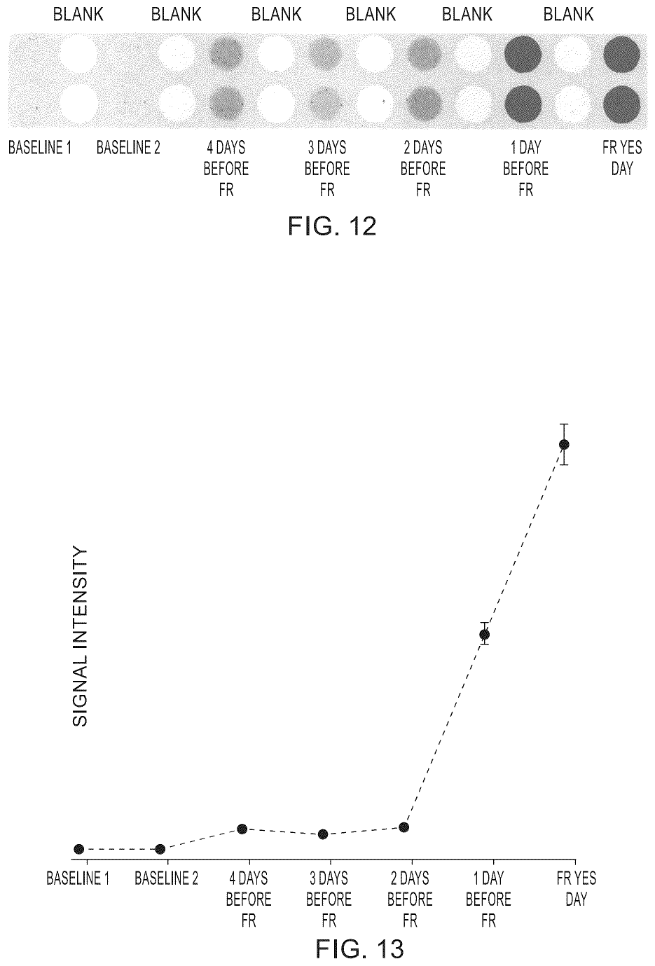

A typical test looks as shown in FIG. 12 were the first and second rows are duplicate samples. A darker circle indicates a higher concentration of hCG in the sample. As would be expected, the baseline days show very low signal, while the days preceding the first response yes day increase in intensity. The image from FIG. 12 is quantified and plotted in FIG. 13, where error bars represent 1 standard deviation.

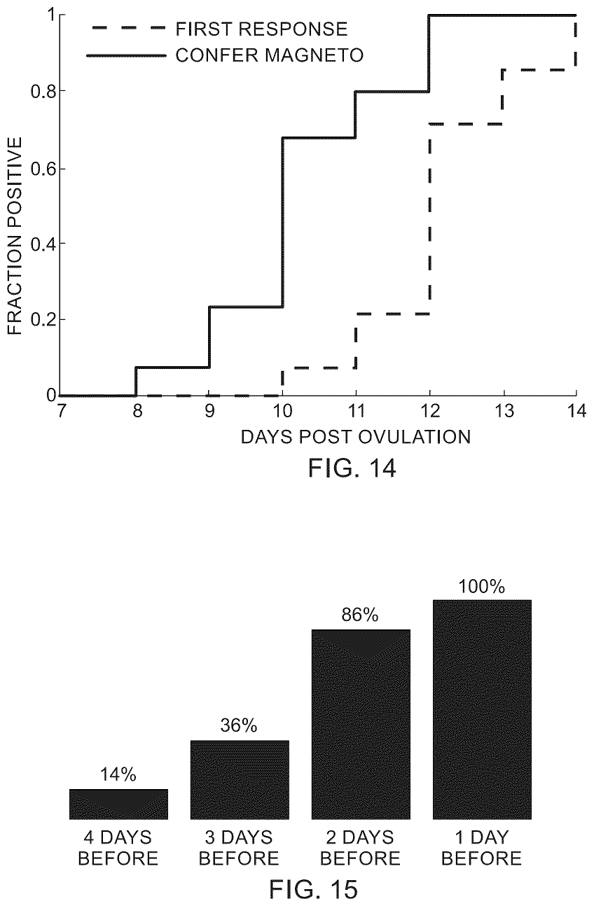

Performance Comparison. FIG. 14 shows a stair-step curve illustrating the fraction positive as a function of days post ovulation (day of LH spike) where the Confer Magneto is the immunoassay described herein.

Baseline Comparison. Each day that is analyzed is compared to the signal of a day's urine that was collected prior to ovulation, also known as a baseline day. This is because there is a characteristic baseline amount of hCG in each person. A yes/no decision is made for pregnancy by calculating the probability that the signal obtained is greater than the largest baseline signal that has been measured for this subject.

A probability is calculated by assuming each day's signal is described by a Gaussian probability with the mean equal to the measured signal and the standard deviation estimates for each respective day. The probability for the baseline day signal, P_{BL}(S), is given by:

$${P}_{B L} ( S ) = \frac{1}{\sqrt{2 π {σ}_{B L}}} {e}^{\frac{- {( S - {μ}_{B L} )}^{2}}{2 {σ}_{BL}^{2}}}$$

With signal (S), a mean (μ_{BL}) and standard deviation (σ_{BL}). The probability for the current day signal, P_{CD} (S), being analyzed is:

$${P}_{C D} ( S ) = \frac{1}{\sqrt{2 π {σ}_{C D}}} {e}^{\frac{- {( S - {μ}_{C D} )}^{2}}{2 {σ}_{CD}^{2}}}$$

With signal (S), a mean (μ_{CD}) and standard deviation (σ_{CD})

The probability that the current day signal is greater than the BL signal is 1 minus the probability that the current day signal is less than or equal to the baseline day signal, P_{CD≤BL}, given by:

$${P}_{C D \leq B L} = {\int }_{- ∞}^{∞} {\int }_{- ∞}^{S} {P}_{B L} ( S ) {P}_{C D} ( {S}^{′} ) {dSdS}^{′}$$

These integrals are numerically estimated. If the difference between any two days is greater than 4(σ_{BL}+σ_{CD}) we let P_{CD≤BL}=0.

P_{CD≥BL}=1−P_{CD≤BL} and thus if the difference between any two days is greater than 4(σ_{BL}+σ_{CD}) we let P_{CD≤BL}=1 (or 100%). These numbers were used in determining the %-certainty in the figure reproduced here for convenience.

A summary of the pregnancy data showing the percent of samples which read positive for a given number of days before First Response is shown in FIG. 15.

Materials and Methods. Provided as set forth in Example 2 (FIGS. 2A to 2F).

### hCG: 2 Phase Binding Protocol—7 Minute Protocol.

### Buffers:

a. CD=Conjugate Diluent (0.1×PBS, 0.5% BSA, 0.05% Sodium Azide)
b. Chon Block is a commercially available blocking solution

### Conjugate Antibodies to Quantum Dots:

Reaction Buffer: 5 mM potassium phosphate, pH 7.4, 0.5% 20K MW PEG.

Procedure:
a. Make 100 mg/mL solutions of EDC and Sulfo-NHS
   i. 2 mg sulfo-NHS+20 uL H₂O.
   ii. 4.8 mg EDC+48 μL H₂O.
b. Add 0.8 μL EDC and 1.6 μL sulfo-NHS to 16.7 μL Quantum Dots (gold core particle with a silica shell impregnated with 100-600 quantum dots (nanoComposix)).
c. Rotate for 30 minutes.
d. Centrifuge 5 minutes at 3600 RCF.
e. Remove supernatant and resuspend with 16.7 μL reaction buffer.
f. Sonicate 10 seconds and vortex to fully resuspend.
g. Resuspend in 17 μL of 1.7 mg/mL 4F9 Antibody.
h. Incubate 1 hour.
i. Add 3.75 μL hydroxylamine, rotate for 10 minutes.
j. Centrifuge for 5 minutes at 3600 RCF, remove supernatant and resuspend with 100 μL reaction buffer.
k. Centrifuge for 5 minutes at 3600 RCF, remove supernatant and resuspend with appropriate volume of conjugate diluent.

### Make Working Solutions:

Mix the following in a 0.5 mL Protein Lo Bind eppendorf tube (solutions may be saved for future use by storing at 4° C.):
a. 1.1 μM 5011_biotin Antibody in Conjugate Diluent
   i. 10 μl of Conjugate Diluent
   ii. 2 μl of Stock (6.6 μM) 5011_biotin Antibody
b. 2.5× dilute 4F9_Quantum Dots
   i. 3.75 μL of Chon Block
   ii. 2.5 μL of Stock 4F9_Quantum Dots

### Magnetic Bead Preparation:

a. Mix the following in a 0.5 mL Protein Lo Bind eppendorf tube:
   i. 4.4 μL 100 nm Stock Streptavidin Magnetic beads.
   ii. 2.2 μL of 1.1 μM 5011_biotin Antibody (In Conjugate Diluent).
b. Incubate at room temperature for 5 minutes on a rotator.
c. Add 4.14 μL of Saturated biotin solution.
d. Mix thoroughly with a pipette.
e. Incubate for an additional 1 minute.
f. Place the tube on the magnet stand and allow it to sit 1 minute (Note: This recipe can be scaled up if preparing beads for a large number of samples once the total volume is over 100 μL the time on the magnet stand needs to be increased for instance a 300 μL volume will require 4 minutes on the magnet stand to fully recover the magnetic beads).
g. Remove the supernatant with a P20 pipette (or appropriate pipette that will remove the volume if scaled up).
h. Add 20 μL of Chon Block to the pellet and mix with a pipette to re-suspend
i. Place the tube on the magnet stand and allow it to sit 1 minute (see note above about timing for volumes >100 μl).
j. Remove the supernatant with a P20 pipette (or appropriate pipette that will remove the volume if scaled up).
k. Add 4.4 μL of Chon Block to the pellet and mix with a pipette to re-suspend.

Method provides 4.4 μL of Prepped Magnetic Beads.

### Phase 1 Binding: Magnetic Bead Analyte Binding

a. Mix the following in a 0.5 mL Protein Lo Bind eppendorf tube:
   i. 4 μL of Prepped Magnetic Beads
   ii. 25 μL of Urine
b. Incubate at room temperature for 1 minute on the bench.
c. Place the tube on the magnet stand and allow it to sit for 45 seconds.
d. Remove the supernatant with a P200 pipette (or appropriate pipette that will remove the volume if scaled up).
e. Add 4 μL of Chon Block to the pellet and mix with a pipette to re-suspend.

Method provides 4 μL of Phase 1 Binding Mix.

### Phase 2 Binding: Quantum Dot Binding

a. Mix the following in a 0.5 mL Protein Lo Bind eppendorf tube:
   i. 4 μL of Phase 1 Binding Mix
   ii. 1 μL of 2.5× dilute 4F9_Quantum Dots
b. Incubate at room temperature for 5 minutes.

Method provides 5 μL of Phase 2 Binding Mix.

### Pulldown and Rinse

a. Place the tube containing 5 μL of Phase 2 Binding Mix on magnet stand and allow it to sit for 20 seconds
b. Keep the tube on the stand for the remaining 5 steps
   i. Remove the supernatant with a P20 pipette (or appropriate pipette that will remove the volume if scaled up).
   ii. Add 100 μL of 1×TBS buffer with a P200 pipette.
   iii. Remove 100 μL of TBS buffer with a P200 pipette.
   iv. Add 100 μL of TBS buffer with a P200 pipette.
   v. Remove 100 μL of TBS buffer with a P200 pipette.
c. Remove tube from the Magnet Stand.
d. Add 10 μL of Conjugate Diluent to the pellet and mix with a P20 pipette to re-suspend.

Method provides 10 μL of analyte bound to both a magnetic particle and a fluorescent Quantum Dot ready to be imaged.

### Example 14—Protocol for Analyzing Urine Samples to Detect Estrone-3-Glucoronide

The following represents an exemplary protocol for analyzing urine samples to detect estrone-3-glucoronide (E3G) according to a method described herein.

The magnetic beads in this assay are magnetic nanoparticles coated with E3G according to the foregoing protocol.

The reporter in this assay is a fluorescently labeled nanoparticle, which has been coated with an antibody (Absolute Antibody 4115) that binds to E3G.

### Equipment Used

The following equipment is used.

CPX2800 Ultrasonic Digital Bench Top Cleaner
Rugged Rotator: #099A-RD4612
SpectraMax i3x
Pipetman P200 Pipette
Pipetman P100 Pipette
Pipetman P20 Pipette
Pipetman P10 Pipette
Promega Magnetic Separation stand (0.5 mL)
Promega Magnetic Seperation stand (0.5 mL)
Timer

### Experimental Procedure

Before the day of collection (e.g., about 5 pm before the day of collection), dilute the magnetic beads and reporters in Chon Block, according the following formulas:

$$Volume of Reporters ( uL ) = Ceiling ( ( \frac{( Number of Subjects ) \times ( 3 Replicates ) \times ( 3 uL per Replicate ) + ( 54 uL for Calibration )}{( 50 X Dilution Factor )} ) \times ( 1.2 X Safety Factor ) )$$

$$Volume of ChonBlock ( uL ) = ( Volume of magnetic beads ) \times ( 50 X Dilution Factor ) - ( Volume of magnetic beads )$$

$$Volume of Magnetic Beads ( uL ) = Ceiling ( ( \frac{( Number of Subjects ) \times ( 3 Replicates ) \times ( 3 uL per Replicate ) + ( 54 uL for Calibration )}{( 21.74 X Dilution Factor )} ) \times ( 1.2 X Safety Factor ) )$$

$$Volume of ChonBlock ( uL ) = ( Volume of Magnetic Beads ) \times ( 21.74 X Dilution Factor ) - ( Volume of Magnetic Beads ) .$$

a. Sonicate reporters by holding the tube in the sonicator for 10 s. Mix reporters using a P100 set to 100.
b. Add a volume of Chon Block to an 0.5 mL Eppendorf Protein Lo-bind tube using an appropriately sized pipette.
c. Add a volume of E3G coupled magnetic beads using a P10 to the Chon Block. Pipette up and down to ensure the pipette has been cleared of Stock magnetic beads.
d. Mix the now-diluted E3G magnetic beads with an appropriately sized pipette
e. Sonicate E3G coupled magnetic beads by holding the tube in the sonicator for 10 s. Mix Magbeads using a P100 set to 100.
f. Add a volume of Chon Block to a 0.5 mL Eppendorf Protein Lo-bind tube using an appropriately sized pipette.
g. Add a volume of E3G coupled magnetic beads using an appropriately sized pipette to the Chon Block. Pipette up and down to ensure the pipette has been cleared of magnetic beads.
h. Mix the now-diluted E3G magnetic beads with an appropriately sized pipette.
i. Store both diluted reagents at in a metal rack at 4° C. Reagents are will be used within 20 hours.

While the urine sample is being collected, allocate 3 μL of the magnetic beads to three 0.5 mL Eppendorf Protein Lo-bind tubes pre-labeled with the subject number, cycle date and replicate number (1,2,3), using a P10 pipette set to 3 μL.

When the sample arrives, add 3 μL of Urine to the First replicate tube.

Immediately after, start the timer, counting up in minutes.

After 30 seconds, with a P10 pipette add 3 μL of reporter to the First replicate tube. Mix with a P10 set to 3 μL.

As the timer hits 1 minute, using a P10 pipette add 3 μL of urine into second replicate tube. Mix with a P10 set to 3 μL.

As 1:30 seconds elapses on the timer, with a P10 pipette add 3 μL of reporter to the second replicate tube. Mix with a P10 set to 3 μL.

As the timer hits 2 minutes, using a P10 pipette add 3 μL of urine into third replicate tube. Mix with a P10 set to 3 μL.

As 2:30 seconds elapses on the timer, with a P10 pipette add 3 μL of reporter to the third replicate tube. Mix with a P10 set to 3 μL.

After 5 minutes elapse on the timer, place the First replicate tube on the Promega magnet stand and check as timer counts up by 1 minute.

At the 6-minute time point, place the second replicate tube on the second Promega magnet stand.

At 6:10, with a P20 Pipette set to 20 μL remove the supernatant from the First replicate tube, while keeping the tube on the magnet stand.

While keeping the tube on the magnet stand, add with a P200 pipette 100 μL of tris buffered saline (TBS) to the First replicate tube, and then remove the 100 μL of TBS by over-pipetting.

While keeping the tube on the magnet stand, add with a P200 pipette another 100 μL of TBS to the First replicate tube, and then remove the 100 μL of TBS by over-pipetting.

Remove the Eppendorf tube from the magnet stand.

With a P20 pipette add 20 μL of TBS, and mix thoroughly by pipetting up and down.

At the 7-minute time point, place the third replicate tube on the First Promega magnet stand.

At 7:10, with a P20 Pipette set to 20 μL remove the supernatant from the second replicate tube, while keeping the tube on the magnet stand.

While keeping the tube on the magnet stand, add with a P200 pipette 100 μL of TBS to the second replicate tube, and then remove the 100 μL of TBS by over-pipetting.

While keeping the tube on the magnet stand, add with a P200 pipette another 100 μL of TBS to the second replicate tube, and then remove the 100 μL of TBS by over-pipetting.

Remove the Eppendorf tube from the magnet stand.

With a P20 pipette add 20 μL of TBS, and mix thoroughly by pipetting up and down.

At 8:10, with a P20 Pipette set to 20 μL remove the supernatant from the second replicate tube, while keeping the tube on the magnet stand.

While keeping the tube on the magnet stand, add with a P200 pipette 100 μL of TBS to the second replicate tube, and then remove the 100 μL of TBS by over-pipetting.

While keeping the tube on the magnet stand, add with a P200 pipette another 100 μL of TBS to the second replicate tube, and then remove the 100 μL of TBS by over-pipetting.

Remove the Eppendorf tube from the magnet stand.

With a P20 pipette add 20 μL of TBS, and mix thoroughly by pipetting up and down.

Store the assay tubes for later analysis.

### Spectra Max Analysis

With a P20 Pipette Load 20 μL of each sample onto the 384-well plate.

With a P10 Pipette Load 20 μL of TBS into 2 wells on the same 384-well plate.

Load the 384-well plate into the SpectraMax and analyze.

### Example 15—Protocol for Analyzing Urine Samples to Detect Human Chorionic Gonadotropin

The following represents an exemplary protocol for analyzing urine samples to detect human chorionic gonadotropin (HCG) according to a method described herein.

The magnetic beads in this assay are magnetic nanoparticles coated with an antibody (Scripps GC099) that binds HCG.

The reporter in this assay is a fluorescently labeled nanoparticle, which has been coated with an antibody (Scripps GC099) that binds HCG.

### Equipment Used

The following equipment is used.

| | |
|---|---|
| Sonicator EQP-00089 | CPX2800 Ultrasonic Digital |
| | Bench Top Cleaner |
| Rotator EQP-00099 | Rugged Rotator: #099A-RD4612 |
| SpectraMax i3x EQP-00001 | SpectraMax i3x |
| P200 Pipette | Pipetman 200 uL FA10005M |
| P100 Pipette | Pipetman 100 uL FA10004M |
| P20 Pipette | Pipetman 20 uL FA10003M |
| P10 Pipette | Pipetman 10 uL FA10002M |
| Promega Magnetic Separation | Z5331 |
| stand (0.5 mL) | |
| Promega Magnetic Seperation | |
| stand (0.5 mL) | |
| Timer | |
| Timer | |
| Corning 384 well Black Clear bottom | |
| Polystyrene | |

### Experimental Procedure

Prior to the assay, dilute the magnetic beads and reporter in Chon Block, according the following recipes:
a. Sonicate reporter by holding the tube in the sonicator for 10 s. Mix reporter using a P100 set to 100.
b. Add 50 μL of Chon Block to an 0.5 mL Eppendorf Protein Lo-bind tube using a P200 set to 50 μL.
c. Add 5 μL magnetic beads to the 50 μL of Chon Block. Pipette up and down to ensure the pipette has been cleared of magnetic beads.
d. Mix the now-diluted magnetic beads with a P200 set to 150.
e. Sonicate magnetic beads by holding the tube in the sonicator for 10 s. Mix magnetic beads using a P100 set to 100.
f. Add 59.4 μL of Chon Block to an 0.5 mL Eppendorf Protein Lo-bind tube using a P200.
g. Add 6 μL of the magnetic beads to the 59.4 μL of Chon Block. Pipette up and down to ensure the pipette has been cleared of magnetic beads.
h. Mix the now-diluted magnetic beads with a P200 set to 125.
i. Store both diluted reagents at in a metal rack at 4° C.

Add 16 μL of urine to three 0.5 mL Eppendorf Protein Lo-bind tubes pre-labeled with the subject number, cycle date and replicate number (1,2,3), using a P20 pipette set to 16 μL.

When the sample arrives, add 2 μL of reporter to the First replicate tube.

Immediately after, start the timer, counting up in minutes.

After 30 seconds, with a P10 pipette add 2 μL of magnetic beads to the First replicate tube. Mix with a P20 set to 16 μL.

As the timer hits 1 minute, using a P10 pipette add 2 μL of reporter into second replicate tube. Mix with a P10 set to 2 μL.

As 1:30 seconds elapses on the timer, with a P10 pipette add 2 μL of magnetic beads to the second replicate tube. Mix with a P10 set to 2 μL.

As the timer hits 2 minutes, using a P10 pipette add 2 μL of reporter into third replicate tube. Mix with a P10 set to 2 μL.

As 2:30 seconds elapses on the timer, with a P10 pipette add 2 μL of magnetic beads to the third replicate tube. Mix with a P10 set to 2 μL.

After 20 minutes elapse on the timer, place the First replicate tube on the Promega magnet stand and check as timer counts up by 1 minute.

At the 21-minute time point, place the second replicate tube on the second Promega magnet stand.

At 21:10, with a P20 Pipette set to 20 μL remove the supernatant from the First replicate tube, while keeping the tube on the magnet stand.

While keeping the tube on the magnet stand, add with a P200 pipette 100 μL of TBS to the First replicate tube, and then remove the 100 μL of TBS by over-pipetting.

While keeping the tube on the magnet stand, add with a P200 pipette another 100 μL of TBS to the First replicate tube, and then remove the 100 μL of TBS by over-pipetting.

Remove the Eppendorf tube from the magnet stand.

With a P20 pipette add 20 μL of TBS, and mix thoroughly by pipetting up and down.

At the 22-minute time point, place the third replicate tube on the First Promega magnet stand.

At 22:10, with a P20 Pipette set to 20 μL remove the supernatant from the second replicate tube, while keeping the tube on the magnet stand.

While keeping the tube on the magnet stand, add with a P200 pipette 100 μL of TBS to the second replicate tube, and then remove the 100 μL of TBS by over-pipetting.

While keeping the tube on the magnet stand, add with a P200 pipette another 100 μL of TBS to the second replicate tube, and then remove the 100 μL of TBS by over-pipetting.

Remove the Eppendorf tube from the magnet stand.

With a P20 pipette add 20 μL of TBS, and mix thoroughly by pipetting up and down.

At 23:10, with a P20 Pipette set to 20 μL remove the supernatant from the second replicate tube, while keeping the tube on the magnet stand.

While keeping the tube on the magnet stand, add with a P200 pipette 100 μL of TBS to the second replicate tube, and then remove the 100 μL of TBS by over-pipetting.

While keeping the tube on the magnet stand, add with a P200 pipette another 100 μL of TBS to the second replicate tube, and then remove the 100 μL of TBS by over-pipetting.

Remove the Eppendorf tube from the magnet stand.

With a P20 pipette add 20 μL of TBS, and mix thoroughly by pipetting up and down.

Store the assay tubes for later analysis.

### Spectramax Analysis

With a P20 Pipette Load 20 μL of each sample onto the 384-well plate.

With a P10 Pipette Load 20 μL of TBS into 2 wells on the same 384-well plate.

Load the 384-well plate into the SpectraMax and analyze.

### Example 16—Protocol for Analyzing Urine Samples to Detect Luteinizing Hormone

The following represents an exemplary protocol for analyzing urine samples to detect luteinizing hormone (LH) according to a method described herein.

The magnetic beads in this assay are magnetic nanoparticles coated with an antibody (Medix 5304) that binds LH.

The reporter in this assay is a fluorescently labeled nanoparticle, which has been coated with an antibody (Medix 5304) that binds LH.

### Equipment Used

The following equipment is used.

| | |
|---|---|
| Sonicator EQP-00089 | CPX2800 Ultrasonic Digital |
| | Bench Top Cleaner |
| Rotator EQP-00099 | Rugged Rotator: #099A-RD4612 |
| SpectraMax i3x EQP-00001 | SpectraMax i3x |
| P200 Pipette | Pipetman 200 μL FA10005M |
| P100 Pipette | Pipetman 100 μL FA10004M |
| P20 Pipette | Pipetman 20 μL FA10003M |
| P10 Pipette | Pipetman 10 μL FA10002M |
| Promega Magnetic Separation | Z5331 |
| stand (0.5 mL) | |
| Promega Magnetic Seperation | |
| stand (0.5 mL) | |
| Timer | |
| Timer | |
| Corning 384 well Black Clear bottom | |
| Polystyrene | |

### Experimental Procedure

Before collection (e.g., about 5 pm the day before collection), dilute the magnetic beads and reporter in Chon Block, according the following recipes:
a. Sonicate reporter by holding the tube in the sonicator for 10 s. Mix reporter using a P100 set to 100.
b. Add 150 μL of ChonBlock to an 0.5 mL Eppendorf Protein Lo-bind tube using a P200 set to 150.
c. Add 3 μL of the magnetic beads to the 150 μL of Chon Block. Pipette up and down to ensure the pipette has been cleared of reporter.
d. Mix the now-diluted magnetic beads with a P200 set to 150.
e. Sonicate magnetic beads by holding the tube in the sonicator for 10 s. Mix magnetic beads using a P100 set to 100.
f. Add 125 μL of Chon Block to an 0.5 mL Eppendorf Protein Lo-bind tube using a P200 set to 125.
g. Add 25 μL magnetic beads to the 125 μL of Chon Block. Pipette up and down to ensure the pipette has been cleared of Stock magnetic beads.
h. Mix the now-diluted magnetic beads with a P200 set to 125.
i. Store both diluted reagents at in a metal rack at 4° C.

While the urine sample is being collected, allocate 3 μL of the magnetic beads to three 0.5 mL Eppendorf Protein Lo-bind tubes pre-labeled with the subject number, cycle date and replicate number (1,2,3), using a P10 pipette set to 3 μL.

When the sample arrives, add 3 μL of Urine to the First replicate tube.

Immediately after, start the timer, counting up in minutes.

After 30 seconds, with a P10 pipette add 3 μL of reporter to the First replicate tube. Mix with a P10 set to 3 μL.

As the timer hits 1 minute, using a P10 pipette add 3 μL of urine into second replicate tube. Mix with a P10 set to 3 μL.

As 1:30 seconds elapses on the timer, with a P10 pipette add 3 μL of reporter to the second replicate tube. Mix with a P10 set to 3 μL.

As the timer hits 2 minutes, using a P10 pipette add 3 μL of urine into third replicate tube. Mix with a P10 set to 3 μL.

As 2:30 seconds elapses on the timer, with a P10 pipette add 3 μL of reporter to the third replicate tube. Mix with a P10 set to 3 μL.

After 5 minutes elapse on the timer, place the First replicate tube on the Promega magnet stand and check as timer counts up by 1 minute.

At the 6-minute time point, place the second replicate tube on the second Promega magnet stand.

At 6:10, with a P20 Pipette set to 20 μL remove the supernatant from the First replicate tube, while keeping the tube on the magnet stand.

While keeping the tube on the magnet stand, add with a P200 pipette 100 μL of TBS to the First replicate tube, and then remove the 100 μL of TBS by over-pipetting.

While keeping the tube on the magnet stand, add with a P200 pipette another 100 μL of TBS to the First replicate tube, and then remove the 100 μL of TBS by over-pipetting.

Remove the Eppendorf tube from the magnet stand.

With a P20 pipette add 20 μL of TBS, and mix thoroughly by pipetting up and down.

At the 7-minute time point, place the third replicate tube on the First Promega magnet stand.

At 7:10, with a P20 Pipette set to 20 μL remove the supernatant from the second replicate tube, while keeping the tube on the magnet stand.

While keeping the tube on the magnet stand, add with a P200 pipette 100 μL of TBS to the second replicate tube, and then remove the 100 μL of TBS by over-pipetting.

While keeping the tube on the magnet stand, add with a P200 pipette another 100 μL of TBS to the second replicate tube, and then remove the 100 μL of TBS by over-pipetting.

Remove the Eppendorf tube from the magnet stand.

With a P20 pipette add 20 μL of TBS, and mix thoroughly by pipetting up and down.

At 8:10, with a P20 Pipette set to 20 μL remove the supernatant from the second replicate tube, while keeping the tube on the magnet stand.

While keeping the tube on the magnet stand, add with a P200 pipette 100 μL of TBS to the second replicate tube, and then remove the 100 μL of TBS by over-pipetting.

While keeping the tube on the magnet stand, add with a P200 pipette another 100 μL of TBS to the second replicate tube, and then remove the 100 μL of TBS by over-pipetting.

Remove the Eppendorf tube from the magnet stand.

With a P20 pipette add 20 μL of TBS, and mix thoroughly by pipetting up and down.

Store the assay tubes for later analysis.

### Spectra Max Analysis

With a P20 Pipette Load 20 μL of each sample onto the 384-well plate.

With a P10 Pipette Load 20 μL of TBS into 2 wells on the same 384-well plate.

Load the 384-well plate into the SpectraMax and analyze.

### Example 17—Protocol for Analyzing Serum Samples to Detect C-Reactive Protein

The following represents an exemplary protocol for analyzing serum samples to detect C-reactive protein (CRP) according to a method described herein.

The magnetic beads in this assay are magnetic nanoparticles coated with an antibody that binds CRP (anti-CRP-C2).

The reporter in this assay is a fluorescently labeled nanoparticle, which has been coated with an antibody (anti-CRP-C6).

### Experimental Procedure

Pre-Incubation of Streptavidin Magnetic Beads with Biotinylated Anti-CRP-C2 Antibody:
a. Mix magnetic beads with antibody, as mentioned above, which is diluted in conjugate diluent to 1 μM, for 5 minutes on rotator.
b. Add saturated biotin solution and incubate the combination for 1 minute.
c. Place on magnet and pulldown, then wash with Chon Block.
d. Pull down again and then resuspend magnetic beads in Chon Block.

### Reporter Preparation

The concentration of a reporter solution for this assay was 440 μM, which was diluted with Chon Block from a stock solution of 2.2 nM.

### Serum Preparation

Serum was prepared by pulling down 20 μL of the aforementioned magnetic beads and then resuspending in 10 μL of serum, then incubating the same for 10 minutes.

Another pull down was performed for 30 seconds and then the supernatant was transferred to a new tube.

### CRP Dilutions

Prepare CRP dilutions in Chon Block from a stock solution of 21.28 mg/mL CRP at concentrations of 2.5 mg/L, 1.25 mg/L, and 0.5 mg/mL.

### Preparation of Spiked Serum Samples

Spike normal serum samples to prepare spiked samples according to the following:
a. 10 mg/L: 8 μL of 2.5 mg/L CRP dilution+2 μL Normal Serum (2 mg/L Final);
b. 5 mg/L: 8 μL of 1.25 mg/L CRP dilution+2 μL Normal Serum (1 mg/L Final);
c. 2 mg/L: 8 μL of 0.5 mg/L CRP dilution+2 μL Normal Serum (0.4 mg/L Final); and
d. 0 mg/L: 8 μL of Chon Block+2 μL Normal Serum (0 mg/L Final).

### Assay Protocol

In a set of four tubes, add the foregoing spiked normal serum samples. Then, preallocate 3 μL of the magnetic beads to each tube. After the magnetic beads, add 3 μL of the sample to be analyzed to each tube. After about 30 seconds, add 3 μL of the reporter and then incubate for about 30 minutes.

After incubation time, pull down sample tubes on a magnetic stand, rinse twice with 100 μL, and resuspend in a final volume of 20 μL TBS.

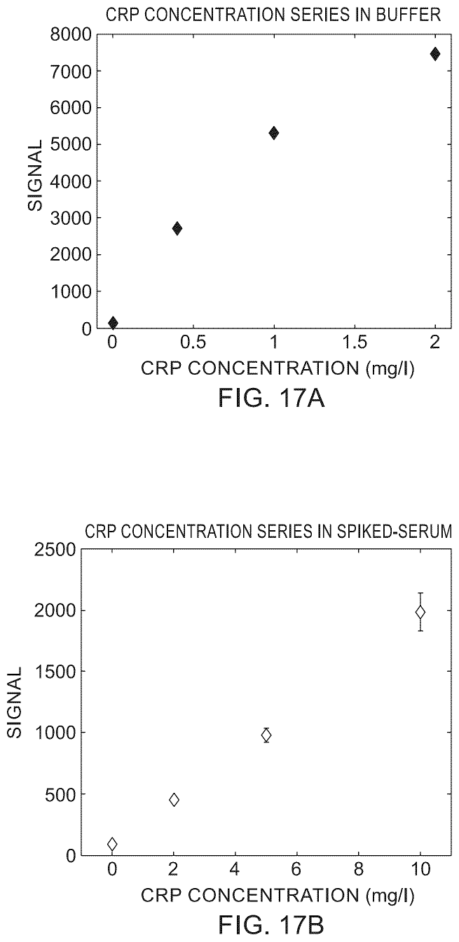

The results of this assay are shown in FIGS. 17A and 17B, which show the CRP concentration series in buffered solution (FIG. 17A) and the CRP concentration in the spiked serum (FIG. 17B).

It is claimed:

1. A method for detecting the presence, absence, or level of an analyte of interest in a sample, the method comprising the steps of:
contacting a sample with a magnetic conjugate comprising a magnetic particle and a capture moiety configured to bind the analyte of interest in the sample;
contacting the sample with a reporter conjugate comprising a reporter and a reporter binding moiety configured to bind the analyte of interest in the sample;
binding the analyte of interest with the capture moiety and the reporter binding moiety;
separating the analyte of interest from the sample by applying a magnetic field to the analysis chamber; and
detecting the presence, absence, or level of the analyte of interest by detecting the reporter.

2. The method of claim 1, wherein the reporter comprises a metal core and a silica shell.

3. The method of claim 2, wherein the silica shell is impregnated with a plurality of quantum dots.

4. The method of claim 2 or 3, wherein the metal core comprises gold.

5. The method of claim 1, wherein the reporter comprises a plurality of quantum dots.

6. The method of any one of the preceding claims, wherein the reporter is a fluorescent reporter, a phosphorescent reporter, or a colorimetric reporter.

7. The method of any one of the preceding claims, further comprising the step of concentrating the analyte of interest in the sample by applying a magnetic field to the analysis chamber after contacting the sample with the magnetic conjugate; and then reducing the volume of the sample in the analysis chamber.

8. The method of claim 7, further comprising the step of deactivating the magnetic field before contacting the sample with the reporter conjugate.

9. The method of any one of the preceding claims, further comprising the steps of concentrating the analyte of interest in the sample by applying a magnetic field to the analysis chamber after contacting the sample with the magnetic conjugate; removing a volume of the sample from the analysis chamber; and adding one or both of a volume of buffer and an additional volume of the sample to the analysis chamber.

10. The method of claim 9, further comprising the step of deactivating the magnetic field before contacting the sample with the reporter conjugate.

11. The method of claim 1, wherein the reporter conjugate is labeled with biotin and the reporter is functionalized with streptavidin.

12. The method of any one of the preceding claims, wherein the analyte of interest is selected from the group consisting of human chorionic gonadotropin (hCG), luteinizing hormone (LH)/Lutropin, prostate specific antigen (PSA), herpes simplex virus (HSV) antibodies, estrone-3-glucuronide (E3G), bacteria, hemoglobin AlC, C-reactive protein, an inflammation biomarker, troponin, lyme disease antigen, lyme disease antibodies, an LDL biomarker, an HDL biomarker, a total cholesterol biomarker, thyroid stimulating hormone, a hepatitis C virus biomarker, a rhino virus biomarker, an influenza virus biomarker, a liver function biomarker, estrogen, progesterone, lactic acid, and combinations thereof.

13. A method for detecting the presence, absence, or level of an analyte of interest in a sample, the method comprising the steps of:
contacting a sample with a magnetic conjugate comprising a magnetic particle and a capture moiety configured to bind the analyte of interest in the sample;
binding the analyte of interest with the capture moiety;
separating the analyte of interest from the sample by applying a magnetic field to the analysis chamber to pull down the magnetic conjugates with analyte of interest associated therewith;
contacting the sample with a reporter conjugate comprising a reporter and a reporter binding moiety configured to bind the analyte of interest in the sample;
binding the analyte of interest with the reporter binding moiety;
separating the analyte of interest with the reporter binding moiety bound thereto from the sample by applying a magnetic field to the analysis chamber; and
detecting the presence, absence, or level of the analyte of interest by detecting the reporter with a light source and photodetector.

14. The method of claim 13, wherein the reporter comprises a fluorescent reporter, a phosphorescent reporter, or a colorimetric reporter.

15. The method of claim 13, wherein the reporter conjugate comprises a plurality of quantum dots.

16. The method of any one of claims 13 to 15, wherein the analyte of interest is selected from the group consisting of human chorionic gonadotropin (hCG), luteinizing hormone (LH)/Lutropin, prostate specific antigen (PSA), herpes simplex virus (HSV) antibodies, estrone-3-glucuronide (E3G), bacteria, hemoglobin AlC, C-reactive protein, an inflammation biomarker, troponin, lyme disease antigen, lyme disease antibodies, an LDL biomarker, an HDL biomarker, a total cholesterol biomarker, thyroid stimulating hormone, a hepatitis C virus biomarker, a rhino virus biomarker, an influenza virus biomarker, a liver function biomarker, estrogen, progesterone, lactic acid, and combinations thereof.

17. A method for detecting the presence, absence, or level of an analyte of interest in a sample, the method comprising the steps of:
contacting a sample with a magnetic conjugate comprising a magnetic particle and a capture moiety configured to bind the analyte of interest in the sample;
binding the analyte of interest with the capture moiety;
contacting the sample with a reporter-labeled analyte configured to bind the magnetic conjugate in the absence of the analyte of interest in the sample;
separating the analyte of interest from the sample by applying a magnetic field to the sample; and
detecting the presence, absence, or level of the analyte of interest by detecting the reporter.

18. The method of claim 17, wherein the reporter comprises a fluorescent reporter, a phosphorescent reporter, or a colorimetric reporter.

19. The method of claim 17 or 18, wherein reporter-labeled analyte comprises a plurality of quantum dots.

20. The method of any one of claims 17 to 19, wherein the analyte of interest is selected from the group consisting of human chorionic gonadotropin (hCG), luteinizing hormone (LH)/Lutropin, prostate specific antigen (PSA), herpes simplex virus (HSV) antibodies, estrone-3-glucuronide (E3G), bacteria, hemoglobin AlC, C-reactive protein, an inflammation biomarker, troponin, lyme disease antigen, lyme disease antibodies, an LDL biomarker, an HDL biomarker, a total cholesterol biomarker, thyroid stimulating hormone, a hepatitis C virus biomarker, a rhino virus biomarker, an influenza virus biomarker, a liver function biomarker, estrogen, progesterone, lactic acid, and combinations thereof.

21. A method for detecting the presence, absence, or level of an analyte of interest in a sample, the method comprising the steps of:
contacting a sample with a reporter conjugate comprising a reporter and a reporter binding moiety configured to bind the analyte of interest in the sample;
binding the analyte of interest with the reporter binding moiety;
contacting the sample with a magnetic particle-labeled analyte configured to bind the reporter conjugate in the absence of the analyte of interest in the sample;
separating the magnetic particle-labeled analyte from the sample by applying a magnetic field to the sample; and
detecting the presence, absence, or level of the analyte of interest by detecting the reporter.

22. The method of claim 21, wherein the reporter comprises a fluorescent reporter, a phosphorescent reporter, or a colorimetric reporter.

23. The method of claim 21 or 22, wherein the reporter conjugate comprises a plurality of quantum dots.

24. The method of any one of claims 21 to 23, wherein the analyte of interest is selected from the group consisting of human chorionic gonadotropin (hCG), luteinizing hormone (LH)/Lutropin, prostate specific antigen (PSA), herpes simplex virus (HSV) antibodies, estrone-3-glucuronide (E3G), bacteria, hemoglobin AlC, C-reactive protein, an inflammation biomarker, troponin, lyme disease antigen, lyme disease antibodies, an LDL biomarker, an HDL biomarker, a total cholesterol biomarker, thyroid stimulating hormone, a hepatitis C virus biomarker, a rhino virus biomarker, an influenza virus biomarker, a liver function biomarker, estrogen, progesterone, lactic acid, and combinations thereof.

25. A method for detecting the presence, absence, or level of an analyte of interest in a sample, the method comprising the steps of:
contacting a sample with a magnetic conjugate comprising a magnetic particle and a capture moiety configured to bind the analyte of interest in the sample;
binding the analyte of interest with the capture moiety;
contacting the sample with a reporter binding moiety comprising a biotin label configured to bind the analyte of interest in the sample;
contacting the sample with a reporter comprising a streptavidin label configured to bind the biotin label;
separating the analyte of interest from the sample by applying a magnetic field to the sample; and
detecting the presence, absence, or level of the analyte of interest by detecting the reporter.

26. The method of claim 25, wherein the reporter comprises a fluorescent reporter, a phosphorescent reporter, or a colorimetric reporter.

27. The method of claim 25 or 26, wherein the reporter comprises a plurality of quantum dots.

28. The method of any one of claims 25 to 27, wherein the analyte of interest is selected from the group consisting of human chorionic gonadotropin (hCG), luteinizing hormone (LH)/Lutropin, prostate specific antigen (PSA), herpes simplex virus (HSV) antibodies, estrone-3-glucuronide (E3G), bacteria, hemoglobin AlC, C-reactive protein, an inflammation biomarker, troponin, lyme disease antigen, lyme disease antibodies, an LDL biomarker, an HDL biomarker, a total cholesterol biomarker, thyroid stimulating hormone, a hepatitis C virus biomarker, a rhino virus biomarker, an influenza virus biomarker, a liver function biomarker, estrogen, progesterone, lactic acid, and combinations thereof.

29. The method of any one of claims 1 to 28, comprising the step of adding the sample to an analysis chamber.

30. The method of claim 29, wherein the step of contacting the sample with the magnetic conjugate comprises contacting the sample with the magnetic conjugate in the analysis chamber.

31. The method of claim 29 or 30, wherein the step of adding the sample to the analysis chamber comprises adding the sample to a sample collector in fluid communication with the analysis chamber.

32. The method of claim 31, wherein the step of contacting the sample with the magnetic conjugate comprises contacting the sample with the magnetic conjugate in the sample collector.

33. A kit for providing the method of any one of claims 1 to 32.

34. A method for evaluating a biological event comprising measuring a biomarker using the method of any one of claims 1 to 32.
